(19) United States
(12) Patent Application Publication
(10) Pub. No.: US 2023/0395237 A1
(43) Pub. Date: Dec. 7, 2023

(54) SYSTEMS AND METHODS FOR IMAGE PROCESSING

(71) Applicant: SHANGHAI UNITED IMAGING HEALTHCARE CO., LTD., Shanghai (CN)

(72) Inventors: Yuan BAO, Shanghai (CN); Xuan XU, Shanghai (CN); Peng WANG, Shanghai (CN); Liyi ZHAO, Shanghai (CN)

(73) Assignee: SHANGHAI UNITED IMAGING HEALTHCARE CO., LTD., Shanghai (CN)

(21) Appl. No.: 18/331,139

(22) Filed: Jun. 7, 2023

(30) Foreign Application Priority Data
Jun. 7, 2022 (CN) 202210633031.5
Jun. 20, 2022 (CN) 202210699687.7

(51) Int. Cl. G16H 30/20; G06T 15/00; G06T 7/20; H04N 5/74

(52) U.S. Cl. CPC G16H 30/20; G06T 15/00; G06T 7/20; H04N 5/74; G06T 2207/20021

(57) ABSTRACT

The present disclosure provides methods and systems for image processing. The methods may include obtaining original projection data of a target subject. For each of at least one slice location of the target subject, the methods may include generating a plurality of candidate slice images of the slice location based on a plurality of distance-weight relationships and the original projection data. Each of the plurality of distance-weight relationships may indicate a weight of a portion of the original projection data acquired by the radiation source and a distance from the radiation source to the slice location when acquiring the portion of the original projection data. The methods may further include generating at least one target medical image of the target subject based on the plurality of candidate slice images of each of the at least one slice location.

### CROSS-REFERENCE TO RELATED APPLICATIONS

This application claims priority to Chinese Patent Application No. 202210699687.7, filed on Jun. 20, 2022, and Chinese Patent Application No. 202210633031.5, filed on Jun. 7, 2022, the contents of each of which are incorporated herein by reference.

### TECHNICAL FIELD

The present disclosure generally relates to the imaging field, and more particularly, relates to systems and methods for image processing.

### BACKGROUND

Medical imaging techniques have been widely used in a variety of fields including, e.g., medical treatments and/or diagnosis. However, during the use of the medical imaging technique, a target subject needs to be scanned for a certain time to acquire scanning data. The target subject always has a motion (e.g., a respiratory motion, a cardiac motion, an intestinal motion, a rigid motion, etc.) during the acquisition of the scanning data, which can result in that a medical image generated based on the scanning data includes motion artifacts, thereby reducing the accuracy of the medical image. Therefore, the scanning data and/or the medical image need to be processed. For example, image deformation algorithms and/or image interpolation algorithms may be used to correct the medical image. However, the image deformation may lead to changes in the overall integral value (e.g., a total brightness value) of the medical image, which results in loss of image information and reduces the accuracy of the medical deformation.

Therefore, it is desirable to provide systems and methods for image processing, which can reduce or eliminate the artifacts in the medical image and/or maintain the overall integral value of the medical image during the image deformation, thereby improving the accuracy of image processing.

### SUMMARY

In an aspect of the present disclosure, a method for image processing is provided. The method may be implemented on a computing device having at least one processor and at least one storage device. The method may include obtaining original projection data of a target subject. For each of at least one slice location of the target subject, the method may include generating a plurality of candidate slice images of the slice location based on a plurality of distance-weight relationships and the original projection data. Each of the plurality of distance-weight relationships may indicate a weight of a portion of the original projection data acquired by the radiation source and a distance from the radiation source to the slice location when acquiring the portion of the original projection data. The method may further include generating at least one target medical image of the target subject based on the plurality of candidate slice images of each of the at least one slice location.

In some embodiments, the plurality of distance-weight relationships of a slice location may be generated by: determining an initial distance-weight relationship corresponding to the slice location; and determining the plurality of distance-weight relationships by translating the initial distance-weight relationship.

In some embodiments, the initial distance-weight relationship corresponding to the slice location may be determined based on at least one of feature information of the slice location or a moving speed of the radiation source with respect to the target subject.

In some embodiments, the translating the initial distance-weight relationship may include determining motion information of the target subject during the acquisition of the original projection data; and translating the initial distance-weight relationship based on the motion information.

In some embodiments, the generating at least one target medical image of the target subject based on the plurality of candidate slice images of each of the at least one slice location may include for each of the at least one slice location, determining a target slice image from the plurality of candidate slice images of the slice location; and generating a target 3D image of the target subject based on the target slice image of each of the at least one slice location.

In some embodiments, the generating at least one target medical image of the target subject based on the plurality of candidate slice images of each of the at least one slice location may include generating a plurality of candidate 3D images of the target subject based on the plurality of candidate slice images of each of the at least one slice location; obtaining an evaluation score of each of the plurality of candidate 3D images by evaluating each of the plurality of candidate 3D images; and determining a target 3D image from the plurality of candidate 3D images based on the plurality of evaluation scores.

In some embodiments, the generating at least one target medical image of the target subject based on the plurality of candidate slice images of each of the at least one slice location may include determining deformation parameters of the target 3D image; generating a preliminary transformed image by processing the target 3D image based on the deformation parameters; determining updated deformation parameters based on the deformation parameters, the count of the updated deformation parameters being determined based on the size of the target 3D image; and generating the at least one target medical image by processing the preliminary transformed image based on the updated deformation parameters.

In some embodiments, the deformation parameters of the target 3D image may be determined using a parameter determination model, and the parameter determination model may be a trained machine learning model.

In another aspect of the present disclosure, a system for image processing is provided. The system may include at least one storage device including a set of instructions; and at least one processor configured to communicate with the at least one storage device. When executing the set of instructions, the at least one processor may be configured to direct the system to perform operations. The operations may include obtaining original projection data of a target subject. For each of at least one slice location of the target subject, the operations may include generating a plurality of candidate slice images of the slice location based on a plurality of distance-weight relationships and the original projection data. Each of the plurality of distance-weight relationships may indicate a weight of a portion of the original projection data acquired by the radiation source and a distance from the radiation source to the slice location when acquiring the portion of the original projection data. The operations may further include generating at least one target medical image of the target subject based on the plurality of candidate slice images of each of the at least one slice location.

In still another aspect of the present disclosure, a method for image processing is provided. The method may be implemented on a computing device having at least one processor and at least one storage device. The method may include determining deformation parameters of a preliminary image. The method may include generating a preliminary transformed image by processing the preliminary image based on the deformation parameters. The method may also include determining updated deformation parameters based on the deformation parameters. The method may further include generating a target transformed image by processing the preliminary transformed image based on the updated deformation parameters.

In some embodiments, the deformation parameters of the preliminary image may be determined using a parameter determination model, and the parameter determination model may be a trained machine learning model.

In some embodiments, the parameter determination model may be generated by obtaining a plurality of training samples, each of the plurality of training samples including a sample image and a sample transformed image corresponding to the sample image; for each of the plurality of training samples, generating predicted deformation parameters by inputting the sample image of the training sample into an initial model, and generating a predicted transformed image by transforming the sample image of the training sample based on the predicted deformation parameters; and generating the parameter determination model by updating the initial model based on the predicted transformed image and the sample transformed image of each of the plurality of plurality of training samples.

In some embodiments, the generating a preliminary transformed image by processing the preliminary image based on the deformation parameters may include generating a plurality of deformation maps based on the deformation parameters and the preliminary image; for each of a plurality of coordinates in an image coordinate system, determining a deformation coordinate of the coordinate based on the plurality of deformation maps; determining a pixel value of each of a plurality of deformation coordinates of the plurality of coordinates based on the preliminary image; and generating the preliminary transformed image based on the plurality of deformation coordinates and their respective pixel values.

In some embodiments, for each coordinate, the determining a deformation coordinate of the coordinate based on the plurality of deformation maps may include determining whether a preset condition is satisfied based on the count of the deformation parameters and the size of the preliminary image; and in response to determining that the preset condition is satisfied, for each coordinate, determining a corresponding deformation map corresponding to the coordinate, and determining the deformation coordinate of the coordinate based on the deformation map corresponding to the coordinate.

In some embodiments, for each coordinate, the determining a deformation coordinate of the coordinate based on the plurality of deformation maps may include determining whether a preset condition is satisfied based on the count of the deformation parameters and the size of the preliminary image; in response to determining that the preset condition is not satisfied, dividing the preliminary image into a plurality of image blocks, each vertex of the plurality of image blocks corresponding to one of the plurality of deformation maps; and for each coordinate, determining the deformation coordinate of the coordinate based on the plurality of image blocks and the plurality of deformation maps.

In some embodiments, for each coordinate, the determining the deformation coordinate of the coordinate based on the plurality of image blocks and the plurality of deformation maps may include determining first coordinates and second coordinates among the plurality of coordinates, each first coordinate being corresponding to a vertex of the plurality of image blocks, the second coordinates being coordinates other than the first coordinates; determining the deformation coordinates of the first coordinates based on the plurality of deformation maps; and determining the deformation coordinate of each second coordinate based on at least part of the deformation coordinates of the first coordinates.

In some embodiments, the count of the updated deformation parameters may be determined based on the size of the preliminary image.

In some embodiments, the determining updated deformation parameters based on the deformation parameters may include determining the updated deformation parameters by performing interpolation operation on the deformation parameters based on the size of the preliminary image.

In some embodiments, the generating a target transformed image by processing the preliminary transformed image based on the updated deformation parameters may include determining a weighting value of each of the updated deformation parameters, the weighting value relating to a proportion of a deformed region in the preliminary image when the preliminary image is transformed based on the updated deformation parameter; and generating the target transformed image by processing the preliminary transformed image based on the weighting value of each of the updated deformation parameters.

In some embodiments, the preliminary image may be obtained by obtaining original projection data of a target subject; for each of at least one slice location of the target subject, generating a plurality of candidate slice images of the slice location based on a plurality of distance-weight relationships and the original projection data, each of the plurality of distance-weight relationships indicating a weight of a portion of the original projection data acquired by the radiation source and a distance from the radiation source to the slice location when acquiring the portion of the original projection data; and generating the preliminary image of the target subject based on the plurality of candidate slice images of each of the at least one slice location.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a block diagram illustrating an exemplary processing device according to some embodiments of the present disclosure;

FIG. 3 is a flowchart illustrating an exemplary process for generating at least one target medical image according to some embodiments of the present disclosure;

FIG. 4A is a schematic diagram illustrating an exemplary distance-weight relationship according to some embodiments of the present disclosure;

FIG. 4B is a schematic diagram illustrating an exemplary process for translating an initial distance-weight relationship according to some embodiments of the present disclosure;

FIGS. 5A-5C are schematic diagrams illustrating exemplary candidate slice images of a slice location according to some embodiments of the present disclosure;

FIG. 6 is a flowchart illustrating an exemplary process for generating a target 3D image of a target subject according to some embodiments of the present disclosure;

FIG. 7 is a flowchart illustrating an exemplary process for generating a target 3D image of a target subject according to some embodiments of the present disclosure;

FIG. 8 is a block diagram illustrating an exemplary processing device according to some embodiments of the present disclosure;

FIG. 9 is a flowchart illustrating an exemplary process for image transformation according to some embodiments of the present disclosure;

FIG. 10 is a flowchart illustrating an exemplary process for generating a parameter determination model according to some embodiments of the present disclosure;

FIG. 11 is a flowchart illustrating an exemplary process for generating a preliminary transformed image according to some embodiments of the present disclosure;

FIG. 12 is a schematic diagram illustrating an exemplary preliminary image according to some embodiments of the present disclosure;

FIG. 13 is a flowchart illustrating an exemplary process for image transformation according to some embodiments of the present disclosure; and

FIG. 14 is a schematic diagram illustrating an exemplary computing device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Provided herein are systems and methods for non-invasive biomedical imaging/treatment, such as for disease diagnostic, disease therapy, or research purposes. In some embodiments, the systems may include an imaging system. The imaging system may include a single modality system and/or a multi-modality system. The term “modality” used herein broadly refers to an imaging or treatment method or technology that gathers, generates, processes, and/or analyzes imaging information of a subject or treatments the subject. The single modality system may include, for example, an ultrasound imaging system, an X-ray imaging system, a computed tomography (CT) system, a magnetic resonance imaging (MRI) system, an ultrasonography system, a positron emission tomography (PET) system, an optical coherence tomography (OCT) imaging system, an ultrasound (US) imaging system, an intravascular ultrasound (IVUS) imaging system, a near-infrared spectroscopy (NIRS) imaging system, or the like, or any combination thereof. The multi-modality system may include, for example, an X-ray imaging-magnetic resonance imaging (X-ray-MRI) system, a positron emission tomography-X-ray imaging (PET-X-ray) system, a single photon emission computed tomography-magnetic resonance imaging (SPECT-MRI) system, a positron emission tomography-computed tomography (PET-CT) system, a C-arm system, a positron emission tomography-magnetic resonance imaging (PET-MR) system, a digital subtraction angiography-magnetic resonance imaging (DSA-MRI) system, etc. It should be noted that the medical system described below is merely provided for illustration purposes, and not intended to limit the scope of the present disclosure.

The term “pixel” and “voxel” in the present disclosure are used interchangeably to refer to an element in an image. In the present disclosure, the term “image” may refer to a two-dimensional (2D) image, a three-dimensional (3D) image, or a four-dimensional (4D) image (e.g., a time series of 3D images). In some embodiments, the term “image” may refer to an image of a region (e.g., a region of interest (ROI)) of a subject. In some embodiment, the image may be a medical image, an optical image, etc.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

The present disclosure relates to systems and methods for image processing. The methods may include obtaining original projection data of a target subject. The original projection data may be acquired when a radiation source is moved to a plurality of locations with respect to the target subject. For each slice location of the target subject, the methods may include generating a plurality of candidate slice images of the slice location based on a plurality of distance-weight relationships and the original projection data. Each of the plurality of distance-weight relationships may indicate a weight of projection data acquired by the radiation source and a distance from the radiation source to the slice location when acquiring the projection data. The methods may further include generating at least one target medical image of the target subject based on the plurality of candidate slice images of each of the at least one slice location.

By introducing the plurality of distance-weight relationships, the plurality of candidate slice images of each slice location may be generated, and a target medical image including no artifacts or minimum artifacts may be generated based on the plurality of candidate slice images, which can reduce the artifacts (e.g., the motion artifacts) included in the target medical image, and improve the image quality of the target medical image.

The present disclosure also provides systems and methods for image transformation. The methods may include determining deformation parameters of a preliminary image (e.g., the target medical image). The methods may include generating a preliminary transformed image by processing the preliminary image based on the deformation parameters. The methods may also include determining updated deformation parameters based on the deformation parameters. The count of the updated deformation parameters may be determined based on the size of the preliminary image. The methods may further include generating a target transformed image by processing the preliminary transformed image based on the updated deformation parameters. In some embodiments, the target transformed image may be generated by introducing a weighting value of each of the updated deformation parameters. Therefore, an overall integral value of the target transformed image may be the same as an overall integral value of the preliminary image, and no further brightness adjustment needs to be performed on the target transformed image, thereby simplifying the process of the image transformation, and improving the efficiency and accuracy of the image transformation.

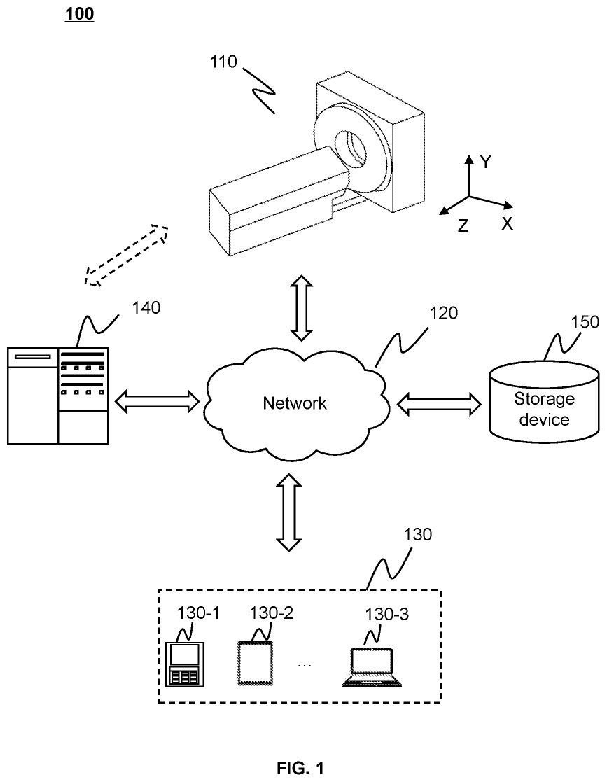

FIG. 1 is a schematic diagram illustrating an exemplary imaging system 100 according to some embodiments of the present disclosure. As shown in FIG. 1, the imaging system 100 may include an imaging device 110, a network 120, one or more terminals 130, a processing device 140, and a storage device 150. In some embodiments, the imaging device 110, the processing device 140, the storage device 150, and/or the terminal(s) 130 may be connected to and/or communicate with each other via a wireless connection (e.g., the network 120), a wired connection, or a combination thereof. The connection between the components in the imaging system 100 may be variable.

The imaging device 110 may be configured to generate or provide image data by scanning a target subject or at least a portion of the target subject. For example, the imaging device 110 may acquire original projection data of the target subject when a radiation source of the imaging device 110 is moved to a plurality of locations with respect to the target subject.

Merely by way of example, the imaging device 110 may be a CT device, and the CT device may include a supporting assembly, a detector assembly, a detection region, a table, and a radiation emitting assembly. The supporting assembly (e.g., a gantry) may support the detector assembly and the radiation emitting assembly. The target subject may be placed on the table and moved to the detection region along with a movement of the table. In some embodiments, the radiation emitting assembly may include one or more radiation sources configured to irradiate the target subject. For example, a radiation source of the radiation emitting assembly may emit a plurality of radiation beams (e.g., X-rays) to the target subject, and the detector assembly may collect image data (e.g., the original projection data) by detecting radiation beams passing through the target subject.

In some embodiments, the radiation source of the imaging device 110 may be moved to a plurality of locations with respect to the target subject during the acquisition of the original projection data. For example, the radiation source of the imaging device 110 may be rotated around the target subject (e.g., rotate along a rail of the supporting assembly) in a scanning angle range. The scanning angle range of the radiation source may refer to an angle range or a span of the angle range that the radiation source rotates around the target subject in a scanning cycle. As another example, the target subject may be fixed on the table and moved along with a movement of the table during the acquisition of the original projection data. In some embodiments, the target subject may be moved in or out the detection region by the table, while the radiation source may rotate around the target subject during the scan. In such cases, the radiation source scans the target subject in a spiral path, and the scan can also be referred to as a helical or spiral scan.

The target subject may include patients or other experimental subjects (e.g., experimental mice or other animals). In some embodiments, the target subject may be a patient or a specific portion, organ, and/or tissue of the patient. For example, the target subject may include the head, the neck, the thorax, the heart, the stomach, a blood vessel, soft tissue, a tumor, nodules, or the like, or any combination thereof. In some embodiments, the target subject may be non-biological. For example, the target subject may include a phantom, a man-made object, etc. The terms “object” and “subject” are used interchangeably in the present disclosure.

In some embodiments, a coordinate system may be provided for the imaging system 100. For illustration purposes, the coordinate system may include an X-axis, a Y-axis, and a Z-axis. The X-axis and the Z-axis shown in FIG. 1 may be horizontal, and the Y-axis may be vertical. As illustrated, a positive X direction along the X-axis may be from the left side to the right side of the imaging device 110 viewed from the direction facing the front of the imaging device 110; a positive Y direction along the Y-axis may be from the lower part (or from the floor where the imaging device 110 stands) to the upper part of the imaging device 110; and a positive Z direction along the Z-axis may be the direction in which the target subject is moved out of a scanning channel (or referred to as a bore) of the imaging device 110.

It should be noted that the provided coordinate system is illustrative, and not intended to limit the scope of the present disclosure. In addition, although the following descriptions discuss through various examples to determine a position of an entity by determining a coordinate of an entity in a certain coordinate system, it should be understood that the position of the entity may be determined by determining a coordinate of the entity in another coordinate system (e.g., a coordinate system that has a known transformation relationship with the certain coordinate system). For the convenience of descriptions, coordinates of an entity along an X-axis, a Y-axis, and a Z-axis in a coordinate system are also referred to as X-coordinates, Y-coordinates, and Z-coordinates of the entity in the coordinate system, respectively.

The network 120 may include any suitable network that can facilitate the exchange of information and/or data for the imaging system 100. In some embodiments, one or more components (e.g., the imaging device 110, the terminal 130, the processing device 140, the storage device 150, etc.) of the imaging system 100 may communicate information and/or data with one or more other components of the imaging system 100 via the network 120. In some embodiments, the network 120 may include one or more network access points.

The terminal(s) 130 may include a mobile device 130-1, a tablet computer 130-2, a laptop computer 130-3, or the like, or any combination thereof. In some embodiments, the mobile device 130-1 may include a smart home device, a wearable device, a mobile device, a virtual reality device, an augmented reality device, or the like, or any combination thereof. In some embodiments, the terminal(s) 130 may include a processing unit, a display unit, a sensing unit, an input/output (I/O) unit, a storage unit, etc. In some embodiments, the terminal(s) 130 may be part of the processing device 140.

The processing device 140 may process data and/or information obtained from one or more components (the imaging device 110, the terminal(s) 130, and/or the storage device 150) of the imaging system 100. For example, the processing device 140 may obtain original projection data of the target subject. For each of at least one slice location of the target subject, the processing device 140 may generate a plurality of candidate slice images of the slice location based on a plurality of distance-weight relationships and the original projection data. The processing device 140 may generate at least one target medical image of the target subject based on the plurality of candidate slice images of each of the at least one slice location. As another example, the processing device 140 may generate a transformed image (e.g., a preliminary transformed image, a target transformed image) based on a preliminary image (e.g., the at least one target medical image, the candidate slice image, etc.). In some embodiments, the processing device 140 may be a single server or a server group. The server group may be centralized or distributed. In some embodiments, the processing device 140 may be local or remote. In some embodiments, the processing device 140 may be implemented on a cloud platform.

In some embodiments, the processing device 140 may be implemented by a computing device. For example, the computing device may include a processor, a storage, an input/output (I/O), and a communication port. In some embodiments, the processing device 140, or a portion of the processing device 140 may be implemented by a portion of the terminal 130.

The storage device 150 may store data/information obtained from the imaging device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. In some embodiments, the storage device 150 may store one or more programs and/or instructions to perform exemplary methods described in the present disclosure.

In some embodiments, the imaging system 100 may include one or more additional components and/or one or more components of the imaging system 100 described above may be omitted. Additionally or alternatively, two or more components of the imaging system 100 may be integrated into a single component. A component of the imaging system 100 may be implemented on two or more sub-components.

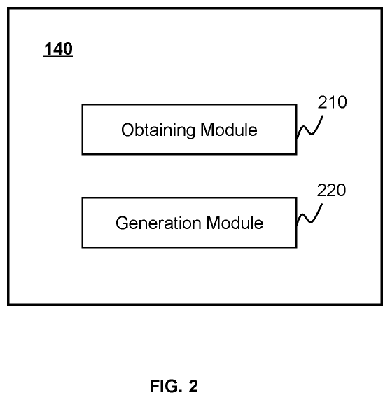

FIG. 2 is a block diagram illustrating an exemplary processing device 140 according to some embodiments of the present disclosure. In some embodiments, the processing device 140 may be in communication with a computer-readable storage medium (e.g., the storage device 150 illustrated in FIG. 1) and execute instructions stored in the computer-readable storage medium. The processing device 140 may include an obtaining module 210 and a generation module 220.

The obtaining module 210 may be configured to obtain original projection data of a target subject. The original projection data may refer to image data acquired by scanning the target subject through an imaging device (e.g., the imaging device 110). In some embodiments, the original projection data may be used to generate (e.g., reconstruct) a medical image of the target subject. More descriptions regarding the obtaining the original projection data of the target subject may be found elsewhere in the present disclosure. See, e.g., operation 302 and relevant descriptions thereof.

The generation module 220 may be configured to, for each of the at least one slice location of the target subject, generate a plurality of candidate slice images of the slice location based on a plurality of distance-weight relationships and the original projection data. For a slice location, each of the plurality of distance-weight relationships may indicate a weight of projection data acquired by the radiation source and a distance from the radiation source to the slice location when acquiring the projection data. More descriptions regarding the generation of the plurality of candidate slice images of the slice location may be found elsewhere in the present disclosure. See, e.g., operation 304 and relevant descriptions thereof.

In some embodiments, the generation module 220 may be further configured to generate at least one target medical image of the target subject based on the plurality of candidate slice images of each of the at least one slice location. The target medical image may refer to a medical image with no artifacts or minimal artifacts. In some embodiments, the target medical image may include a three-dimensional (3D) medical image of the target subject or a target slice image of a slice location of the target subject. More descriptions regarding the generation of the at least one target medical image of the target subject may be found elsewhere in the present disclosure. See, e.g., operation 306 and relevant descriptions thereof.

In some embodiments, the processing device 140 may include one or more other modules, one or more modules mentioned above can be omitted. For example, the processing device 140 may include a storage module to store data generated by the modules in the processing device 140. In some embodiments, any two of the modules may be combined as a single module, and any one of the modules may be divided into two or more units. For example, the generation module 220 may include a first generation unit and a second generation unit, wherein the first generation unit may be configured to, for each of the at least one slice location of the target subject, generate the plurality of candidate slice images of the slice location based on the plurality of distance-weight relationships and the original projection data, and the second generation unit may be configured to generate the at least one target medical image of the target subject based on the plurality of candidate slice images of each of the at least one slice location.

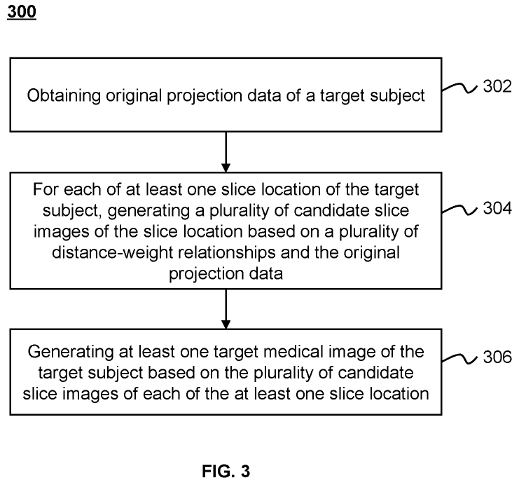

FIG. 3 is a flowchart illustrating an exemplary process for generating at least one target medical image according to some embodiments of the present disclosure. Process 300 may be implemented in the imaging system 100 illustrated in FIG. 1. For example, the process 300 may be stored in the storage device 150 in the form of instructions (e.g., an application), and invoked and/or executed by the processing device 140.

When an imaging device (e.g., the imaging device 110) scans a target subject, scanning data may be acquired within a certain acquisition time. The target subject often has a motion (e.g., a respiratory motion, a cardiac motion, an intestinal motion, a rigid motion, etc.) during the acquisition of the scanning data, which can result in that a medical image generated (e.g., reconstructed) based on the scanning data includes motion artifacts. The motion artifacts may be represented in the form of overlapping and/or blurring, strip artifacts, contour displacements, appendage-like objects, etc., in the medical image. Therefore, the accuracy of the medical image and the diagnosis may be reduced, which brings difficulties in subsequent operations (e.g., automatic lesion detection, computer-aided diagnosis, 3D reconstruction, etc.) based on the medical image, thereby restricting the development of medical imaging techniques. In order to reduce and/or eliminate the motion artifacts in the medical image and improve the accuracy of the medical image, the process 300 may be performed.

In 302, the processing device 140 (e.g., the obtaining module 210) may obtain original projection data of a target subject.

The original projection data may refer to image data acquired by scanning the target subject through an imaging device (e.g., the imaging device 110). In some embodiments, the original projection data may be used to generate (e.g., reconstruct) a medical image of the target subject.

In some embodiments, the target subject may include at least one slice location. A slice location may refer to a sectional plane of the target subject. Exemplary sectional planes may include a cross-section plane, a coronal plane, a sagittal plane, or the like, or any combination thereof. In some embodiments, the slice location refers to a cross-section plane perpendicular to the Z-axis as shown in FIG. 1.

In some embodiments, the original projection data may be acquired when a radiation source (e.g., the radiation source of the imaging device 110) is moved to a plurality of locations with respect to the target subject. For example, the radiation source of the imaging device 110 may move (e.g., rotate around the target subject) and/or the target subject may move (e.g., be translated along the Z-axis direction by a table) during the acquisition of the original projection data.

Correspondingly, the original projection data may include a plurality of sets of projection data. Each of the plurality of sets of projection data may correspond to an acquisition moment of the set of projection data and/or a location of the radiation source when collecting the set of projection data. In some embodiments, a location of the radiation source may be represented by a distance between the radiation source (or a gantry where the radiation source is mounted) and a specific slice location of the target subject along the Z-axis direction. For example, the original projection data may be represented by {P1, P2, P3, . . . , Pt}, wherein Pi refers to a set of projection data collected at time i when the radiation source is located at a certain distance away from a specific slice location along the Z-axis.

In some embodiments, the processing device 140 may obtain the original projection data from an imaging device (e.g., the imaging device 110) or a storage device (e.g., the storage device 150, a database, or an external storage) that stores the original projection data of the target subject.

Since the original projection data is acquired with a certain acquisition time, the target subject may have a motion (e.g., a respiratory motion, a cardiac motion, an intestinal motion, a rigid motion, etc.) during the acquisition of the original projection data. For example, if the target subject includes the chest, a respiratory motion and/or a cardiac motion may occur during the acquisition of the original projection data. As another example, if the target subject includes the abdomen, a respiratory motion and/or an intestinal motion may occur during the acquisition of the original projection data. Therefore, motion correction needs to be performed to reduce or eliminate artifacts (e.g., motion artifacts) in image reconstruction.

In 304, for each of the at least one slice location of the target subject, the processing device 140 (e.g., the generation module 220) may generate a plurality of candidate slice images of the slice location based on a plurality of distance-weight relationships and the original projection data.

For a slice location, each of the plurality of distance-weight relationships may indicate a weight of projection data (e.g., a portion of the original projection data) acquired by the radiation source and a distance from the radiation source to the slice location when acquiring the projection data. The weight may indicate a confidence coefficient corresponding to the projection data acquired by the radiation source. The larger the weight, the higher the confidence coefficient corresponding to the projection data. As described above, the radiation source may move and the distance between the radiation source and the slice location may change during the scan. A distance-weight relationship may also indicate the weight of different sets of projection data collected at different acquisition moments during the scan.

The distance from the radiation source to the slice location may be a distance between a first point (e.g., a surface point, a central point on a surface of the radiation source, a central point, any interior point) of the radiation source and a second point (e.g., a boundary point, a central point, any interior point) of the slice location, a distance between the first point of the radiation source and a surface of the slice location, etc. Merely for illustration, the distance from the radiation source (e.g., the central point of the radiation source) to the slice location may be the distance between the radiation source and the slice location along the Z-axis direction illustrated in FIG. 1. For instance, the distance from the radiation source to the slice location may be represented through a bed code. The processing device 140 may determine a first bed code corresponding to the radiation source (e.g., the first point of the radiation source) and a second bed code corresponding to the slice location (e.g., the second point of the radiation source, the surface of the slice location, etc.), and determine a code difference between the first bed code and the second bed code. The processing device 140 may designate the code difference as the distance from the radiation source to the slice location.

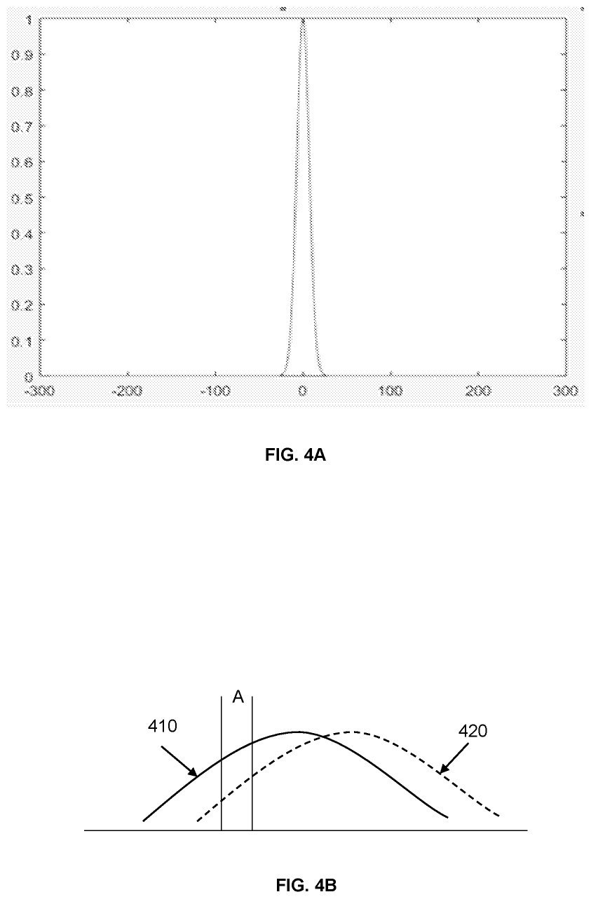

In some embodiments, a distance-weight relationship may be represented as a table, a diagram, a mathematic function, a curve, a model, etc., or any form that can indicate the relationship between the weight of the projection data acquired by the radiation source and the distance from the radiation source to the slice location when acquiring the projection data. For example, referring to FIG. 4A, a distance-weight relationship may be represented as a Gaussian function curve. As illustrated in FIG. 4A, an abscissa axis may represent the distance from the radiation source to the slice location, and an ordinate axis may represent the weight of the projection data acquired by the radiation source. A value “0” on the abscissa axis may represent that the distance from the radiation source to the slice location is 0. The less the distance from the radiation source to the slice location is (e.g., the closer the radiation source is to the slice location), the larger the weight may be. When the distance from the radiation source to the slice location is 0, the weight may have a maximum value (e.g., close to 1).

In some embodiments, for each slice location, the processing device 140 may determine an initial distance-weight relationship corresponding to the slice location.

The initial distance-weight relationship may be regarded as a basic distance-weight relationship that is used to generate the plurality of distance-weight relationships.

In some embodiments, the at least one slice location of the target subject may correspond to a same initial distance-weight relationship. That is, the plurality of distance-weight relationships corresponding to each slice location may be determined based on the same initial distance-weight relationship. For example, the initial distance-weight relationship corresponding to each slice location may be represented as a Gaussian function curve with a same shape (e.g., the Gaussian function curve illustrated in FIG. 4A).

In some embodiments, the at least one slice location of the target subject may correspond to different initial distance-weight relationships. For example, a first slice location of the at least one slice location may correspond to a first initial distance-weight relationship, a second slice location of the at least one slice location may correspond to a second initial distance-weight relationship, and the first initial distance-weight relationship may be different from the second initial distance-weight relationship. The first and second initial distance-weight relationships may be represented by Gaussian function curves with different shapes.

In some embodiments, the initial distance-weight relationship corresponding to the slice location may be determined based on feature information of the slice location, a moving speed of the radiation source with respect to the target subject, or the like, or any combination thereof. The feature information of the slice location may indicate whether the slice location is moved during the acquisition of the original projection data and/or a motion degree of the slice location during the acquisition of the original projection data. For example, the feature information of the slice location may include position information of the slice location, motion information of the slice location, an organ type of the slice location, or the like, or any combination thereof. Merely by way of example, if the feature information of the slice location indicates that the slice location is moved during the acquisition of the original projection data and/or the motion degree during the acquisition of the original projection data exceeds a motion threshold, a half-width of the initial distance-weight relationship (e.g., the Gaussian function illustrated in FIG. 4A) may be decreased. The half-width may indicate an equivalent temporal resolution of an image of the slice location to be reconstructed. If the feature information of the slice location indicates that the slice location is still during the acquisition of the original projection data and/or the motion degree during the acquisition of the original projection data does not exceed the motion threshold, the half-width of the initial distance-weight relationship may be increased. For instance, if the feature information of the slice location indicates that the slice location is still during the acquisition of the original projection data, the processing device 140 may designate a horizontal line (e.g., a constant function) as the initial distance-weight relationship of the slice location.

The moving speed of the radiation source with respect to the target subject may include a rotation speed of the radiation source, a moving speed of the target subject, a moving speed of a bed where the target subject is located (or fixed), etc. Merely by way of example, if the moving speed of the radiation source with respect to the target subject exceeds a speed threshold, the half-width of the initial distance-weight relationship (e.g., the Gaussian function illustrated in FIG. 4A) may be decreased. If the moving speed of the radiation source with respect to the target subject does not exceed the speed threshold, the half-width of the initial distance-weight relationship may be increased. If the moving speed of the radiation source with respect to the target subject is 0, the processing device 140 may designate a horizontal line (e.g., a constant function) as the initial distance-weight relationship of the slice location.

In some embodiments, the initial distance-weight relationship corresponding to the slice location may be determined based on a system default setting or set manually by a user (e.g., a technician, a doctor, a physicist).

By determining different initial distance-weight relationships for different slice locations, actual condition(s) (e.g., specificities of the slice locations) of the scan may be considered into the motion correction, which can improve the suitability between the initial distance-weight relationship and the slice location, thereby improving the efficiency and accuracy of the artifact correction.

In some embodiments, for each slice location, the processing device 140 may determine the plurality of distance-weight relationships by translating the initial distance-weight relationship of the slice location. For example, if the initial distance-weight relationship is represented by the Gaussian function curve illustrated in FIG. 4A, the processing device 140 may randomly translate the Gaussian function curve along the abscissa axis direction by a distance to determine the plurality of distance-weight relationships. The distance may include any suitable distance, for example, −100 millimeters (mm), −80 mm, −50 mm, −20 mm, −10 mm, −5 mm, −2 mm, −1 mm, −0.5 mm, 0 mm, 0.5 mm, 1 mm, 2 mm, 5 mm, 10 mm, 20 mm, 50 mm, 80 mm, 100 mm, etc. By translating the Gaussian function curve multiple times with different distances, multiple Gaussian function curves (i.e., multiple distance-weight relationships) can be obtained.

As another example, the processing device 140 may determine motion information of the target subject during the acquisition of the original projection data. The motion information may indicate a motion state of the target subject. For example, the motion information may include a motion amplitude, a motion direction, etc., of the slice location (e.g., each point of the slice location) at each moment during the acquisition of the original projection data. In some embodiments, the motion information may be obtained using a motion detection device. Exemplary motion detection devices may include a laser radar, a millimeter wave radar, an infrared sensor, or the like, or any combination thereof. After the motion information of the target subject is determined, the processing device 140 may translate the initial distance-weight relationship based on the motion information. Specifically, the initial distance-weight relationship may be translated such that a weight of projection data acquired when the target subject has an obvious motion is reduced. Therefore, the weight of the projection data acquired when the target subject has the obvious motion may be reduced, which may reduce a contribution rate of the projection data acquired when the target subject has the obvious motion, thereby reducing the possibility of the motion artifacts in a reconstructed medical image of the slice location and improving the accuracy of the reconstructed medical image of the slice location. In some embodiments, the initial distance-weight relationship may be included in the plurality of distance-weight relationships.

Merely by way of example, referring to FIG. 4B, if the target subject includes the chest, and the motion information indicates that an obvious respiratory motion occurs when the distance between the slice location and the radiation source is within an interval A. Therefore, an initial distance-weight relationship 410 may be translated rightwards to obtain a distance-weight relationship 420, so as to reduce a weight of the projection data acquired when the distance is within the interval A. Thus, a contribution rate of the projection data acquired when the distance is within the interval A may be reduced, thereby reducing the possibility of the motion artifacts in a reconstructed medical image of the slice location.

A candidate slice image of a slice location may be a slice image that is generated based on the original projection data and a distance-weight relationship. In some embodiments, each of the at least one slice location may correspond to the plurality of candidate slice images, and each of the plurality of candidate slice images may correspond to one of the plurality of distance-weight relationships corresponding to the slice location. For example, the plurality of candidate slice images of the slice location may be regarded as a series of images in a chronological order. That is, the plurality of candidate slice images of the slice location may be regarded as slice images of the slice location acquired at different moments.

Merely by way of example, a plurality of distance-weight relationships corresponding to a slice location may be ordered in a chronological order according to an abscissa corresponding to the maximum value of the weight of each of the plurality of distance-weight relationships, wherein the abscissa corresponding to the maximum value of the weight of the distance-weight relationship indicates a moment corresponding to the distance-weight relationship. For example, a distance-weight relationship corresponding to a minimum abscissa may be regarded as corresponding to a starting moment, and a distance-weight relationship corresponding to a maximum abscissa may be regarded as corresponding to an end moment. Alternatively, a distance-weight relationship corresponding to a maximum abscissa may be regarded as corresponding to a starting moment, and a distance-weight relationship corresponding to a minimum abscissa may be regarded as corresponding to an end moment.

In some embodiments, for each slice location, the processing device 140 may generate the plurality of candidate slice images of the slice location by processing the original projection data based on the plurality of distance-weight relationships corresponding to the slice location.

For example, for a slice location, a candidate slice image of the slice location may be determined based on one of the distance-weight relationships corresponding to the slice location. Specifically, the processing device 140 may obtain a set of weighted projection data of the slice location by performing a weighted summation operation on the sets of projection data of the original projection data based on the distance-weight relationship. In the weighted summation operation, the weight of each set of projection data may be determined based on the distance between the radiation source and the slice location when collecting the set of projection data by looking up the distance-weight relationship. Merely by way of example, as aforementioned, the original projection data may be represented by {P1, P2, P3, . . . , Pt}, wherein Pi refers to a set of projection data collected at time i when the radiation source is located at a certain distance away from a specific slice location along the Z-axis. The weight of each of P1, P2, P3, . . . , Pt may be determined based on its corresponding distance and the distance-weight relationship, and a set of weighted projection data may be determined by performing a weighted summation operation on P1, P2, P3, . . . , Pt. Since there are multiple distance-weight relationships corresponding to the slice location, multiple sets of weighted projection data corresponding to the slice location can be determined.

Then, the processing device 140 may generate the plurality of candidate slice images of the slice location by reconstructing the sets of weighted projection data corresponding to the slice location using an image reconstruction algorithm. Exemplary image reconstruction algorithms may include a back-projection algorithm, an iterative algorithm, an analytical algorithm (e.g., a filtered back-projection algorithm, a Fourier transformation algorithm, etc.), or the like, or any combination thereof. Since the plurality of candidate slice images correspond to different distance-weight relationships, artifacts (e.g., motion artifacts) included in the plurality of candidate slice images may be different.

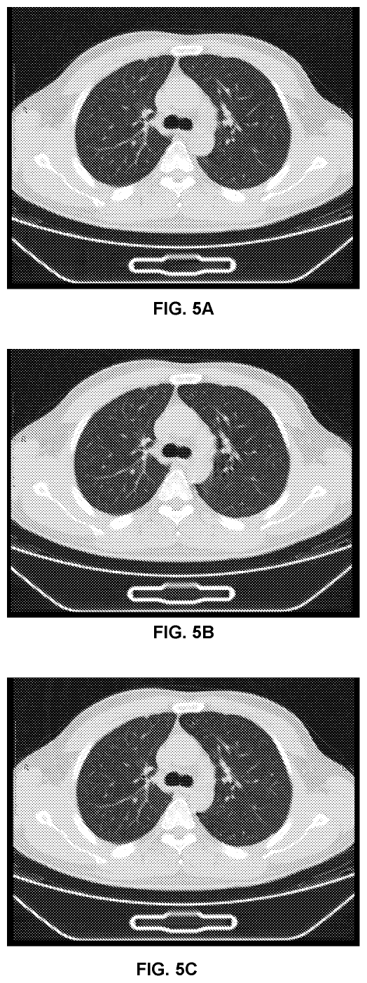

Merely by way of example, referring to FIGS. 5A-5C, FIGS. 5A-5C are schematic diagrams illustrating exemplary candidate slice images of a slice location according to some embodiments of the present disclosure. A candidate slice image in FIG. 5A is generated based on a distance-weight relationship, which is determined by translating an initial distance-weight relationship leftwards. A candidate slice image in FIG. 5B is generated based on a distance-weight relationship without translating the initial distance-weight relationship. That is, the candidate slice image in FIG. 5B is generated based on the initial distance-weight relationship. A candidate slice image in FIG. 5C is generated based on a distance-weight relationship, which is determined by translating the initial distance-weight relationship rightwards. The three candidate slice images may be regarded as slice images of the slice location acquired at three moments. As illustrated in FIGS. 5A-5C, different candidate slice images include different degrees of motion artifacts.

In 306, the processing device 140 (e.g., the generation module 220) may generate at least one target medical image of the target subject based on the plurality of candidate slice images of each of the at least one slice location.

The target medical image may refer to a medical image with no artifacts or minimal artifacts. For example, the target medical image may be a medical image that satisfies an artifact requirement of the user or a medical image that can be used for diagnosis. In some embodiments, the target medical image may include a three-dimensional (3D) medical image of the target subject or a target slice image of a slice location of the target subject.

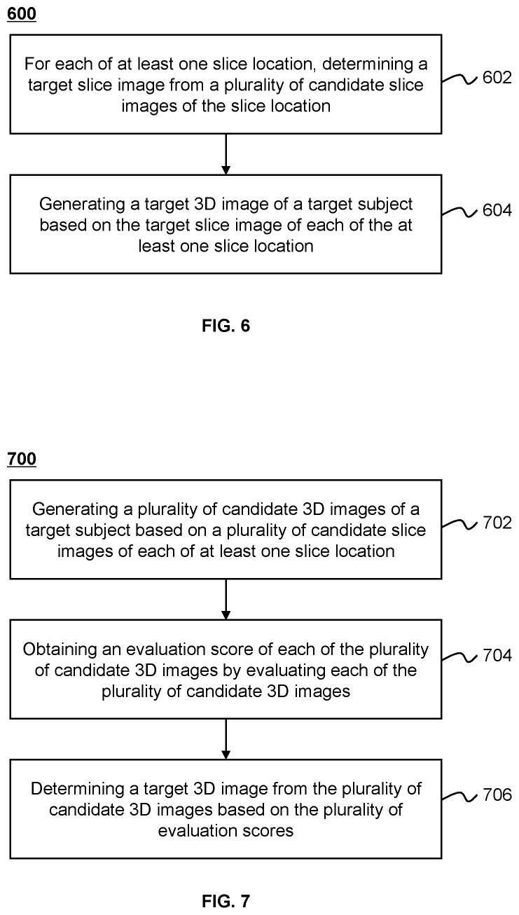

In some embodiments, the at least one target medical image may include a target 3D image, and the processing device 140 may generate the target 3D image of the target subject based on the plurality of candidate slice images of each of the at least one slice location. For example, the processing device 140 may determine a target slice image from the plurality of candidate slice images of the slice location for each of the at least one slice location, and generate the target 3D image of the target subject based on the target slice image of each of the at least one slice location. As another example, the processing device 140 may generate a plurality of candidate 3D images of the target subject based on the plurality of candidate slice images of each of the at least one slice location, and obtain an evaluation score of each of the plurality of candidate 3D images by evaluating each of the plurality of candidate 3D images. The processing device 140 may determine the target 3D image from the plurality of candidate 3D images based on the plurality of evaluation scores. More descriptions regarding the generation of the target 3D image of the target subject may be found in elsewhere in the present disclosure (e.g., FIGS. 6 and 7, and the descriptions thereof).

In some embodiments, the processing device 140 may directly generate the plurality of candidate 3D images based on the plurality of distance-weight relationships and the original projection data, and determine the at least one target medical image (e.g., the target 3D image) of the target subject based on the plurality of candidate 3D images. For example, the processing device 140 may obtain the evaluation score of each of the plurality of candidate 3D images by evaluating each of the plurality of candidate 3D images, and determine the target 3D image from the plurality of candidate 3D images based on the plurality of evaluation scores. The evaluation score of each of the plurality of candidate 3D images may be obtained in a similar manner as how an evaluation score is determined as described in FIG. 7.

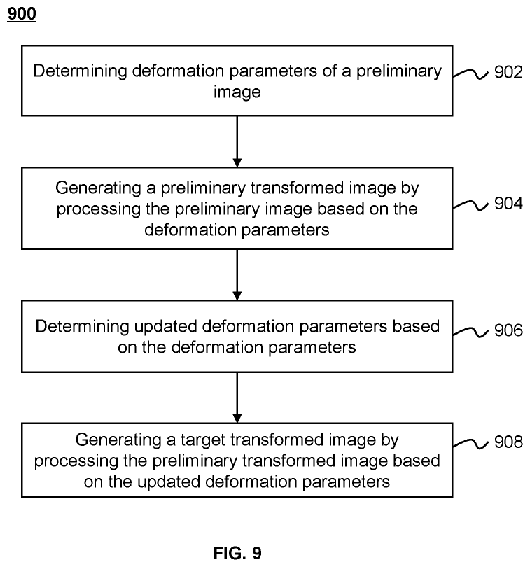

In some embodiments, the processing device 140 may post-process the target 3D image to obtain the at least one target medical image. Exemplary post-processing operations may include image deformation, image enhancement, image denoising, image smoothing, or the like, or any combination thereof. Merely by way of example, the processing device 140 may determine deformation parameters of the target 3D image, generate a preliminary transformed image by processing the target 3D image based on the deformation parameters, determine updated deformation parameters based on the deformation parameters, and generate a target transformed image (i.e., the at least one target medical image) by processing the preliminary transformed image based on the updated deformation parameters. More descriptions regarding the image deformation may be found in elsewhere in the present disclosure (e.g., FIGS. 9-13 and the descriptions thereof).

In some embodiments, the processing device 140 may display the medical images (e.g., the candidate slice image, the target 3D image, the candidate 3D image, the preliminary transformed image, the target transformed image, etc.) on a display interface for a user to view and/or adjust.

According to some embodiments of the present disclosure, by introducing the plurality of distance-weight relationships, the plurality of candidate slice images of each slice location may be generated. Correspondingly, the target medical image including no artifacts or minimum artifacts may be generated based on the plurality of candidate slice images, which can reduce the artifacts (e.g., the motion artifacts) included in the target medical image, and improve the image quality of the target medical image.

FIG. 6 is a flowchart illustrating an exemplary process 600 for generating a target 3D image of a target subject according to some embodiments of the present disclosure. In some embodiments, the process 600 may be performed to achieve at least part of operation 306 as described in connection with FIG. 3.

In 602, for each of at least one slice location of a target subject, the processing device 140 (e.g., the generation module 220) may determine a target slice image from a plurality of candidate slice images of the slice location.

The target slice image of a slice location may refer to a 2D image with no artifacts or minimal artifacts determined based on (e.g., selected from) the candidate slice images of the slice location.

In some embodiments, since artifacts (e.g., motion artifacts) in each of the plurality of candidate slice images are different, a candidate slice image with no artifacts or minimal artifacts may be determined as the target slice image corresponding to the slice location by comparing the plurality of candidate slice images. For example, the processing device 140 may determine an image difference between each two adjacent candidate slice images among the plurality of candidate slice images, and determine an image parameter value of each of the plurality of candidate slice images based on the image differences. The image parameter value of each candidate slice image may indicate an artifact intensity of the candidate slice image. For example, for a candidate slice image, the processing device 140 may determine the image parameter value of the candidate slice image based on the image difference(s) corresponding to the candidate slice image. Merely by way of example, the higher the image difference(s) corresponding to the candidate slice image, the higher the image parameter value of the candidate slice image. The processing device 140 may then determine the target slice image from the plurality of candidate slice images based on the image parameter value of each of the plurality of candidate slice images. For example, the processing device 140 may determine a candidate slice image with a minimum image parameter value as the target slice image.

Merely by way of example, if a plurality of candidate slice images corresponding to a slice location include an image 1, an image 2, an image 3, an image 4, and an image 5 arranged in a chronological order according to an abscissa corresponding to the maximum value of the weight of each of the plurality of distance-weight relationships, the processing device 140 may determine a first image difference between the image 1 and the image 2, a second image difference between the image 2 and the image 3, a third image difference between the image 3 and the image 4, and a fourth image difference between the image 4 and the image 5. The processing device 140 may determine an image parameter value of each of the plurality of candidate slice images based on the image differences (i.e., the first to fourth image differences), and determine a candidate slice image with the minimum image parameter value as the target slice image. For example, if values of the first image difference, the second image difference, the third image difference, and the fourth image difference are increased, the image 1 may be determined as the target slice image. As another example, if values of the first image difference, the second image difference, the third image difference, and the fourth image difference are decreased, the image 5 may be determined as the target slice image. As still another example, if a value of the second image difference is minimum, the image 2 may be determined as the target slice image when the first image difference is less than the third image difference. Alternatively, the image 3 may be determined as the target slice image when the first image difference is larger than the third image difference. As yet another example, if the value of the second image difference is minimum, and the first image difference is equal to the third image difference, either the image 2 or the image 3 may be determined as the target slice image. As yet another example, for each candidate slice location, the processing device 140 may determine an average image difference based on the image difference(s) between the candidate slice location and its adjacent slice location(s). The processing device 140 may determine that the candidate slice location corresponding to the maximum average value has the maximum image parameter value, and the candidate slice location corresponding to the minimum average value has the minimum image parameter value. The processing device 140 may determine the image corresponding to the minimum image parameter value as the target slice image.

In some embodiments, since the artifacts in each of the plurality of candidate slice images are different, motion information of the target subject in the plurality of candidate slice images may be different. Therefore, the processing device 140 may determine the target slice image based on the motion information of the target subject in the plurality of candidate slice images. For example, the processing device 140 may determine the motion information of the target subject in each of the plurality of candidate slice images. The motion information may include a motion direction, a motion amplitude, etc. The processing device 140 may determine the target slice image from the plurality of candidate slice images based on the motion information of each of the plurality of candidate slice images. For example, for each of the plurality of candidate slice images, the processing device 140 may generate a corrected slice image by correcting the candidate slice image based on the motion information of the candidate slice image, and determine an image parameter value of the corrected slice image. The processing device 140 may further determine a corrected slice image corresponding to a minimum image parameter value as the target slice image. The correction may be performed according to a motion correction algorithm, a trained machine learning model, etc., which are not limited herein.

Merely by way of example, for each of the plurality of candidate slice images, the processing device 140 may generate the corrected slice image using a motion artifact correction model. For example, the processing device 140 may obtain the motion artifact correction model, and generate the corrected slice image by inputting each of the plurality of candidate slice images into the motion artifact correction model. Further, the processing device 140 may determine the target slice image based on the plurality of the corrected slice images. The motion artifact correction model may be obtained by training a first initial model based on a plurality of first training samples, each of the plurality of first training samples including a sample slice image of a sample slice location and a sample corrected slice image. In some embodiments, the input of the motion artifact correction model may further include the motion information of the candidate slice image. Correspondingly, each of the plurality of first training samples may further include sample motion information of the sample slice image.

As another example, for each of the plurality of candidate slice images, the processing device 140 may generate the target slice image using a slice image prediction model. For example, the processing device 140 may obtain the slice image prediction model, and generate the target slice image by inputting the plurality of candidate slice images into the slice image prediction model. The slice image prediction model may be obtained by training a second initial model based on a plurality of second training samples, each of the plurality of second training samples including a plurality of sample slice images of a sample slice location and a sample target slice image. In some embodiments, the input of the motion artifact correction model may further include the motion information of each of the plurality of candidate slice images. Correspondingly, each of the plurality of second training samples may further include sample motion information of each of the plurality of sample slice images.

In 604, the processing device 140 (e.g., the generation module 220) may generate a target 3D image of the target subject based on the target slice image of each of the at least one slice location.

For example, the processing device 140 may stack the target slice image of each of the at least one slice location (e.g., along the Z direction) to obtain the target 3D image of the target subject.

According to some embodiments of the present disclosure, for each of at least one slice location of a target subject, the target slice image of the slice location with minimum motion artifact may be determined based on the plurality of candidate slice images of the slice location, thereby reducing or eliminating the artifacts (e.g., the motion artifacts) in the target 3D image generated based on the target slice image of each of the at least one slice location, and improving the image quality of the target 3D image.

FIG. 7 is a flowchart illustrating an exemplary process 700 for generating a target 3D image of a target subject according to some embodiments of the present disclosure. In some embodiments, the process 700 may be performed to achieve at least part of operation 306 as described in connection with FIG. 3.

In 702, the processing device 140 (e.g., the generation module 220) may generate a plurality of candidate 3D images of a target subject based on a plurality of candidate slice images of each of at least one slice location.

The candidate 3D image may refer to a 3D image that is generated based on the candidate slice images of the target subject.

In some embodiments, the plurality of candidate 3D images may be generated by combining at least part of the plurality of candidate slice images of each of the at least one slice location. For example, for each of the at least one slice location, the processing device 140 may randomly determine a candidate slice image from the candidate slice images of the slice location. The processing device 140 may then generate the candidate 3D image by stacking the candidate slice image of each of the at least one slice location.

Merely by way of example, if the target subject includes five slice locations, and each of the five slice locations corresponds to 10 candidate slice images, the processing device 140 may randomly determine a candidate slice image of each of the five slice locations, and generate a candidate 3D image based on the determined candidate slice images. Correspondingly, the processing device 140 may generate the plurality of candidate 3D images. The maximum count of the candidate 3D images may be 10⁵. A count of the plurality of candidate 3D images may be determined based on a system default setting or set manually by a user (e.g., a technician, a doctor, a physicist, etc.).

In 704, the processing device 140 (e.g., the generation module 220) may obtain an evaluation score of each of the plurality of candidate 3D images by evaluating each of the plurality of candidate 3D images.

In some embodiments, the processing device 140 may determine a preset evaluation condition, and evaluate each of the plurality of candidate 3D images based on the preset evaluation condition. The preset evaluation condition may include evaluation parameter(s) to evaluate the image quality of the plurality of candidate 3D images. Exemplary evaluation parameters of a candidate 3D image may include an anatomical definition (e.g., the definition of anatomical structures) in the candidate 3D image, a contrast of critical portions in the candidate 3D image, a uniformity of image signals in the candidate 3D image, an image noise level of the candidate 3D image, an artifact reduction degree of the candidate 3D image, or the like, or any combination thereof. The anatomical definition may be the definition of the texture and boundaries of portions (e.g., the anatomical structures) in the candidate 3D image. For example, if the candidate 3D image is a brain image, the anatomical structure may be anatomical structures (e.g., the brain, the diencephalon, the cerebellum, the brainstem, etc.) of the brain. As another example, if the candidate 3D image is a kidney image, the anatomical structure may be anatomical structures (e.g., the renal cortex, the renal medulla, etc.) of the kidney. The contrast of critical portions in the candidate 3D image may be the contrast between light areas and dark areas in the candidate 3D image, such as, the contrast between the white of the brightest area and the black of the darkest area in different brightness levels. The critical portion may be one or more parts of the target subject that need to be observed, such as, the skull of the brain, lung windows in the chest, soft tissues in the abdomen, etc. The uniformity of the image signals may be a uniformity degree of the image signals obtained when an imaging device (e.g., the imaging device 110) scans the target subject. The image noise level may be a proportion of interference information in the candidate 3D image. For example, some portion of the candidate 3D image may include isolated noise points. The artifact reduction degree may be a degree to which artifacts in the candidate 3D image have been eliminated or reduced.

In some embodiments, for a candidate 3D image, the processing device 140 may determine one or more sub-evaluation scores of the candidate 3D image based on the one or more evaluation parameters in the preset evaluation condition. Each of the one or more sub-evaluation scores may be determined based on one evaluation parameter. For example, if the evaluation parameter has a positive relationship to the image quality of the candidate 3D image, a higher sub-evaluation score may be determined if the evaluation parameter has a higher value.

Further, the processing device 140 may determine the evaluation score of the candidate 3D image based on the one or more sub-evaluation scores of the candidate 3D image. For example, the processing device 140 may determine the evaluation score by determining a sum of the one or more sub-evaluation scores. As another example, the processing device 140 may determine the evaluation score by determining a weighted sum of the sub-evaluation score(s) based on a weighting value of each evaluation parameter. In some embodiments, the weighting value may be determined based on medical experience or other manners, which is not limited herein. For example, weighting values of the anatomical definition, the contrast of critical portions, and the uniformity of image signals may be 0.4, 0.5, and 0.3, respectively.

In 706, the processing device 140 (e.g., the generation module 220) may determine a target 3D image from the plurality of candidate 3D images based on the plurality of evaluation scores.

For example, the processing device 140 may determine a candidate 3D image with a maximum evaluation score as the target 3D image.

According to some embodiments of the present disclosure, the plurality of candidate 3D images of the target subject may be generated based on the plurality of candidate slice images of each of the at least one slice location. Since artifacts (e.g., motion artifacts) in the plurality of candidate slice images are different, artifacts (e.g., motion artifacts) in the plurality of candidate 3D images may be different. Therefore, by obtaining the evaluation score of each of the plurality of candidate 3D images according to the preset evaluation condition (e.g., the evaluation parameters), and determining the target 3D image from the plurality of candidate 3D images based on the plurality of evaluation scores, the candidate 3D image with no artifacts or minimal artifacts may be determined as the target 3D image, thereby reducing or eliminating the artifacts (e.g., the motion artifacts) in the target 3D image, and improving the image quality of the target 3D image.

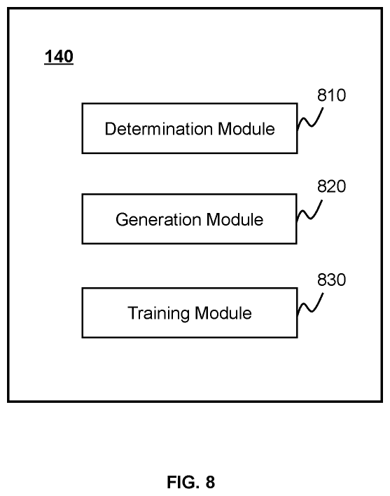

FIG. 8 is a block diagram illustrating an exemplary processing device 140 according to some embodiments of the present disclosure. The modules illustrated in FIG. 8 may be implemented on the processing device 140. In some embodiments, the processing device 140 may be in communication with a computer-readable storage medium (e.g., the storage device 150 illustrated in FIG. 1) and execute instructions stored in the computer-readable storage medium. The processing device 140 may include a determination module 810, a generation module 820, and a training module 830.

The determination module 810 may be configured to determine deformation parameters of a preliminary image. The preliminary image may include any a medical image that needs to be transformed. A deformation parameter may be used to specify how to perform a deformation operation on at least a portion of the preliminary image. More descriptions regarding the determination of the deformation parameters of the preliminary image may be found elsewhere in the present disclosure. See, e.g., operation 902 and relevant descriptions thereof.

The generation module 820 may be configured to generate a preliminary transformed image by processing the preliminary image based on the deformation parameters. The preliminary transformed image may refer to an image that has been preliminarily transformed based on the deformation parameters, and might be further processed. More descriptions regarding the generation of the preliminary transformed image may be found elsewhere in the present disclosure. See, e.g., operation 904 and relevant descriptions thereof.

In some embodiments, the determination module 810 may be further configured to determine updated deformation parameters based on the deformation parameters. The count of the updated deformation parameters may be determined based on the size of the preliminary image. More descriptions regarding the determination of the updated deformation parameters may be found elsewhere in the present disclosure. See, e.g., operation 906 and relevant descriptions thereof.

In some embodiments, the generation module 820 may be further configured to generate a target transformed image by processing the preliminary transformed image based on the updated deformation parameters. The target transformed image may be regarded as the final transformed image. More descriptions regarding the generation of the target transformed image may be found elsewhere in the present disclosure. See, e.g., operation 908 and relevant descriptions thereof.

The training module 830 may be configured to generate one or more machine learning models used for image transformation, such as, a parameter determination model, etc. In some embodiments, the training module 830 may be implemented on the processing device 140 or a processing device other than the processing device 140. In some embodiments, the training module 830 and other modules (e.g., the determination module 810, the generation module 820) may be implemented on a same processing device (e.g., the processing device 140). Alternatively, the training module 830 and other modules (e.g., the determination module 810 and/or the generation module 820) may be implemented on different processing devices. For example, the training module 830 may be implemented on a processing device of a vendor of the machine learning model(s), while the other modules may be implemented on a processing device of a user of the machine learning model(s).

In some embodiments, the determination module 810, the generation module 820, and the training module 830 may be implemented by the generation module 220. For example, the determination module 810, the generation module 820, and the training module 830 may be sub-modules of the generation module 220.

FIG. 9 is a flowchart illustrating an exemplary process 900 for image transformation according to some embodiments of the present disclosure.

Image processing operations (e.g., image registration, motion simulation, motion correction, video processing, photographic image processing, etc.) may be performed based on image deformation and/or image interpolation algorithms. At present, the image interpolation algorithm is always used to perform the interpolation operation by performing a linear or nonlinear combination of values of pixel points around a target pixel point. Therefore, the pixel value after the interpolation is not equal to a sum of the pixel values participating in the interpolation. Further, for the image deformation performed based on the image interpolation algorithm, an overall integral value of a deformed image is different from an overall integral value of a preliminary image corresponding to the deformed image. An overall integral value of an image may refer to a sum of a gray value of each pixel point in the image. Correspondingly, an image brightness of the deformed image is different from an image brightness of the preliminary image, which may reduce the accuracy of the deformed image, and limit the application of the deformed image. For example, for a coronary CT image, the changes in the image brightness may result in a difference between CT values of the preliminary image and CT values of the deformed image, which reduces the accuracy of the deformed image and the medical diagnosis. Therefore, the image brightness of the deformed image needs to be adjusted through a further operation, which complicates the image processing process and reduces the applicability of the image deformation. In order to improve the efficiency and accuracy of the image deformation, the process 900 may be performed.

For illustration purposes, the image deformation of the medical images may be taken as an example. It should be noted that the process 900 may be used to transform any images. For example, images in daily sceneries, biomedical images, radar images, astronomical observation images, geological exploration images, etc., may be transformed according to the process 900.

In 902, the processing device 140 (e.g., the determination module 810) may determine deformation parameters of a preliminary image.

The preliminary image may include any a medical image that needs to be transformed. For example, the preliminary image may be an image reconstructed based on scan data of a target subject. In some embodiments, the preliminary image may include a candidate slice image generated in operation 304, a candidate 3D image, a target 3D image, or a target medical image generated in operation 306, etc.

In some embodiments, the processing device 140 may obtain the preliminary image from an imaging device (e.g., the imaging device 110, etc.) or a storage device (e.g., the storage device 150, a database, or an external storage) that stores the preliminary image of a target subject.

A deformation parameter may be used to specify how to perform a deformation operation on at least a portion of the preliminary image. For example, the deformation parameter may include an affine transformation matrix or a non-affine transformation matrix. The affine transformation matrix may include parameters relating to affine transformation to be performed on at least a portion of the preliminary image. Exemplary affine transformation may include image scaling, image translation, image rotation, image shear, image reflection, image perspective projection, or the like, or any combination thereof. The non-affine transformation matrix may include parameters relating to non-affine transformation to be performed on a portion of the preliminary image. The non-affine transformation may include nonlinear transformation, etc.

In some embodiments, the processing device 140 may determine the deformation parameters of the preliminary image according to processing requirements. The processing requirements may relate to a processing mode, a processing efficiency, a processing capacity, a processing accuracy, etc. The processing mode may include an overall deformation mode or a partition deformation mode for the preliminary image. In the overall deformation mode, one deformation parameter or multiple same deformation parameters may be used to perform the image deformation on the whole preliminary image. In the partition deformation mode, the preliminary image may be divided into a plurality of image blocks, and the plurality of image blocks may be processed through a plurality of deformation parameters that are not completely the same. In some embodiments, in the partition deformation mode, deformation parameters may be set for a portion of the plurality of image blocks. That is, image deformation may be performed on the portion of the plurality of image blocks, while other portion(s) of the plurality of image blocks may be kept from being deformed.

In some embodiments, the partition deformation mode may be used to deform the preliminary image, and the number of the deformation parameters may be equal to the number of the vertex of image blocks of the preliminary image. In other words, a deformation parameter may be determined for each vertex image block of the preliminary image. The deformation parameters of different vertexes may be the same or different. In some embodiments, at least two vertexes may have different deformation parameters, and these vertexes may be deformed in different forms in image deformation.

Merely by way of example, referring to FIG. 12, FIG. 12 is a schematic diagram illustrating an exemplary preliminary image according to some embodiments of the present disclosure. As illustrated in FIG. 12, a height and a width of the preliminary image may be 5, respectively. For example, the preliminary image may be divided into 4 image blocks. The 4 image blocks (as indicated by solid lines) may include 9 vertexes. Correspondingly, 9 deformation parameters may be set for the preliminary image. As another example, the preliminary image may be divided into 16 image blocks. The 16 image blocks may include 25 vertexes. Correspondingly, 25 deformation parameters may be set for the preliminary image.

In some embodiments, the processing device 140 may determine the deformation parameters of the preliminary image using a parameter determination model. For example, the processing device 140 may input the preliminary image into the parameter determination model, and the parameter determination model may output the deformation parameters.

In some embodiments, the parameter determination model may include a trained machine learning model, which can generate the deformation parameters corresponding to the preliminary image. Exemplary parameter determination models may include a generative adversarial network (GAN), a U-net, a pixel recurrent neural network (PixelRNN), a draw network, a variational autoencoder (VAE), or the like, or any combination thereof.

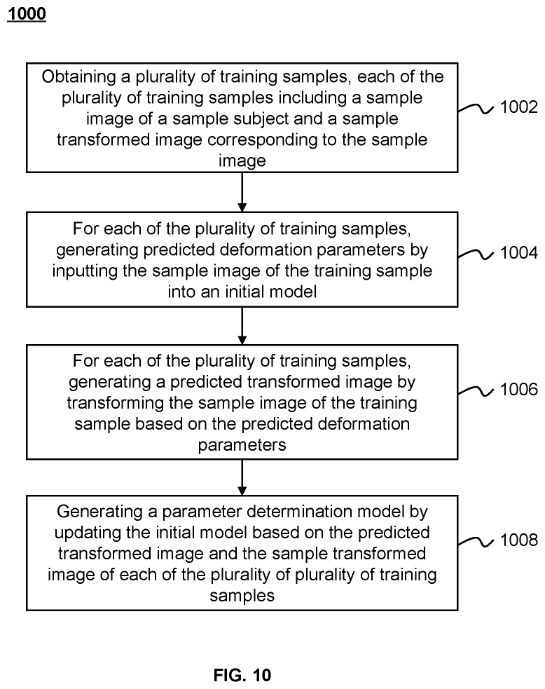

In some embodiments, the parameter determination model may be generated by the processing device 140 or another computing device by training an initial model using a plurality of training samples. Each of the plurality of training samples may include a sample image and a sample transformed image corresponding to the sample image. More descriptions regarding the generation of the parameter determination model may be found in elsewhere in the present disclosure (e.g., FIG. 10 and the descriptions thereof). In some embodiments, the parameter determination model may be previously generated and stored in a storage device (e.g., the storage device 150). The processing device 140 may retrieve the parameter determination model from the storage device and use the parameter determination model to generate the deformation parameters.

In some embodiments, under an ideal condition, the larger the count of the deformation parameters is, the better the deformation effect may be. For example, more vertexes of image blocks may be deformed separately when more deformation parameters are determined. However, in a practical application, the count of the deformation parameters needs to be determined based on actual requirements (e.g., a processing complexity of the preliminary image, a processing capacity of the processing device 140, etc.). Moreover, in the partition deformation processing, the count of the deformation parameters may need to be set according to a size of the preliminary image to achieve the partitioning effect of the preliminary image. By introducing the parameter determination model, the deformation parameters may be generated automatically while considering the actual requirements, which can improve the efficiency and accuracy of the generation of the deformation parameters, and smooth the process of the image transformation.

In 904, the processing device 140 (e.g., the generation module 820) may generate a preliminary transformed image by processing the preliminary image based on the deformation parameters.

The preliminary transformed image may refer to an image that has been preliminarily transformed based on the deformation parameters, and might be further processed.

In some embodiments, the preliminary transformed image may be generated by processing the preliminary image based on the deformation parameters and an interpolation algorithm. Exemplary interpolation algorithms may include a bilinear interpolation algorithm, a nearest neighbor interpolation algorithm, a thin plate spline interpolation, a bicubic interpolation algorithm, or the like, or any combination thereof.

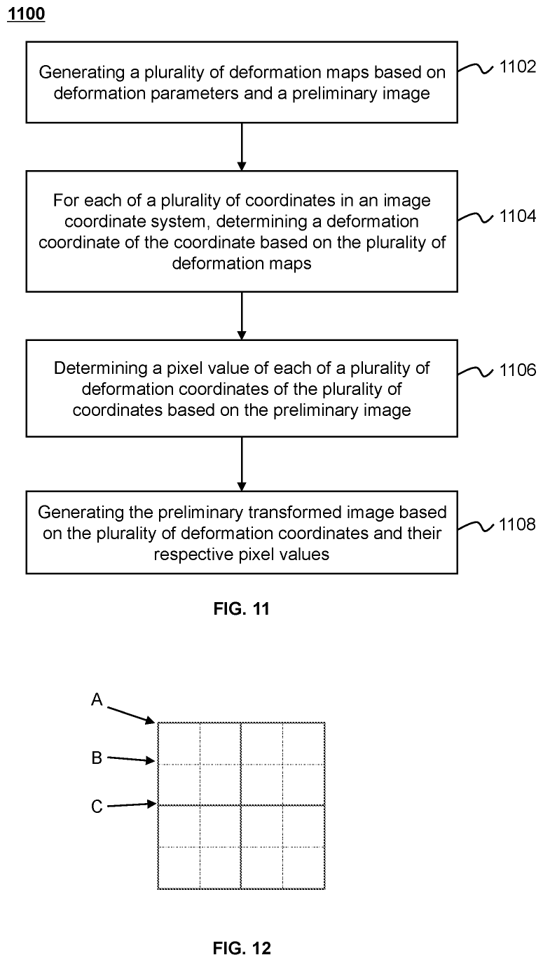

Merely by way of example, the processing device 140 may generate a plurality of deformation maps based on the deformation parameters and the preliminary image. For each of a plurality of coordinates in an image coordinate system, the processing device 140 may determine a deformation coordinate of the coordinate based on the plurality of deformation maps. The processing device 140 may then determine a pixel value of each of a plurality of deformation coordinates of the plurality of coordinate based on the preliminary image. The processing device 140 may further generate the preliminary transformed image based on the plurality of deformation coordinates and their respective pixel values. For example, the processing device 140 may use the interpolation algorithm based on the plurality of deformation coordinates and their respective pixel values to generate the preliminary transformed image. More descriptions regarding the generation of the preliminary transformed image may be found in elsewhere in the present disclosure (e.g., FIG. 11 and the descriptions thereof).

In 906, the processing device 140 (e.g., the determination module 810) may determine updated deformation parameters based on the deformation parameters.

The count of the updated deformation parameters may be determined based on the size of the preliminary image. For example, the count of the updated deformation parameters may be equal to the size of the preliminary image.

In some embodiments, the processing device 140 may determine the updated deformation parameters by performing interpolation operation on the deformation parameters based on the size of the preliminary image. Merely by way of example, if the count of deformation parameters is m×n, and the height and width of the preliminary image (e.g., a 2D image) are H and W, respectively, the processing device 140 may perform the interpolation operation (e.g., the bilinear interpolation algorithm) on the deformation parameters based on the size of the preliminary image to determine the updated deformation parameters. The count of the updated deformation parameters may be H×W. As used herein, m, n, H, and W are positive integers, and a product of m and n is different from a product of H and W. As another example, if the count of deformation parameters is m×n×l, and the length, height, and width of the preliminary image (e.g., a 3D image) are L, H, and W, respectively, the processing device 140 may perform the interpolation operation (e.g., the bilinear interpolation algorithm) on the deformation parameters based on the size of the preliminary image to determine the updated deformation parameters. The count of the updated deformation parameters may be L×H×W. As used herein, m, n, l, L, H, and W are positive integers, and a product of m, n, and l is different from a product of L, H, and W. At this point, each of the updated deformation parameters may correspond to one coordinate in the preliminary image or one coordinate in the preliminary transformed image. In other words, each pixel in the preliminary image or the preliminary transformed image may correspond to one updated deformation parameter.

In 908, the processing device 140 (e.g., the generation module 820) may generate a target transformed image by processing the preliminary transformed image based on the updated deformation parameters.

The target transformed image may be regarded as the final transformed image.

In some embodiments, the processing device 140 may determine a weighting value of each of the updated deformation parameters. The weighting value may relate to a proportion of a deformed region in the preliminary image when the preliminary image is transformed based on the updated deformation parameter, a proportion of an integral value of the preliminary image and an integral value determined after the preliminary image is transformed based on the updated deformation parameter, a proportion of a brightness value of the preliminary image and a brightness value determined after the preliminary image is transformed based on the updated deformation parameter, etc. As described above, each pixel in the preliminary image or the preliminary transformed image may have a corresponding updated deformation parameter. The weighting value of an updated deformation parameter may also be regarded as a weighting value of a pixel in the preliminary image or the preliminary transformed image corresponding to the updated deformation parameter.

In some embodiments, for each of the updated deformation parameters, the processing device 140 may determine a determinant value of the updated deformation parameter, and designate the determinant value as a weighting value of a pixel corresponding to the updated deformation parameter. In some embodiments, the processing device 140 may further arrange the plurality of the determinant values according to a configuration of the pixels in the preliminary image or the preliminary transformed image to determine a determinant matrix corresponding to the preliminary image or the preliminary transformed image.

In some embodiments, the processing device 140 may generate the target transformed image by processing the preliminary transformed image based on the weighting value of each of the updated deformation parameters. For example, for each pixel in the preliminary transformation image, the processing device 140 may multiply the pixel value of the pixel in the preliminary transformed image by the weighting value of an updated deformation parameter corresponding to the pixel, so as to generate the target transformed image. As another example, the preliminary transformed image may be represented as an image matrix, the weighting values of the updated deformation parameters may be represented as a weighting matrix (i.e., the determinant matrix), and the target transformed image may be generated by multiplying the image matrix with the weighting matrix.

By introducing the weighting values, the integral value of the target transformed image may be the same as the integral value of the preliminary image. Therefore, no brightness adjustment needs to be performed on the target transformed image, which may simplify the process of the image transformation, and improve the efficiency and accuracy of the image transformation.

According to some embodiments of the present disclosure, the deformation parameters of the preliminary image may be automatically determined using the parameter determination model, which improves the efficiency and accuracy of the deformation parameter determination, and facilitates the process of the image transformation. The preliminary transformed image may be generated based on the deformation parameters, and the deformation parameters may be updated using the interpolation algorithm to obtain the updated deformation parameters. Further, the target transformed image may be generated by processing the preliminary transformed image based on the updated deformation parameters. The integral value of the target transformed image may be the same as the integral value of the preliminary image, which needs no further brightness adjustment, thereby simplifying the process of the image transformation, and improving the efficiency and accuracy of the image transformation.

FIG. 10 is a flowchart illustrating an exemplary process 1000 for generating a parameter determination model according to some embodiments of the present disclosure. In some embodiments, the process 1000 may be performed to achieve at least part of operation 902 as described in connection with FIG. 9.

In 1002, the processing device 140 (e.g., the training module 830) may obtain a plurality of training samples. Each of the plurality of training samples may include a sample image of a sample subject and a sample transformed image corresponding to the sample image. The sample image may be a medical image of the sample subject before transformation. The sample transformed image may be used as a training label.

In some embodiments, the sample transformed image corresponding to the sample image in the training sample may be generated or confirmed manually. For example, a user may transform the sample image to generate the sample transformed image via a user terminal that displays the sample image. Alternatively, the sample transformed image corresponding to the sample image in the training sample may be an image obtained by performing a motion correction (e.g., a motion texture correction) on the sample image, a high quality image obtained by scanning the sample subject when the sample subject is under a desired state (e.g., a heart rate of the sample subject is well controlled), etc.

In 1004, for each of the plurality of training samples, the processing device 140 (e.g., the training module 830) may generate predicted deformation parameters by inputting the sample image of the training sample into an initial model.

The initial model may be a machine learning model before being trained. Exemplary initial models may include a generative adversarial network (GAN), a U-net, a pixel recurrent neural network (PixelRNN), a draw network, a variational autoencoder (VAE), or the like, or any combination thereof.

For example, the processing device 140 may input the sample image into the initial model, and the initial model may output the predicted deformation parameters.

In 1006, for each of the plurality of training samples, the processing device 140 (e.g., the training module 830) may generate a predicted transformed image by transforming the sample image of the training sample based on the predicted deformation parameters.

In some embodiments, the transformation of the sample image of a training sample may be performed by the initial model. For example, the initial model may include a transformation layer that can generate the predicted transformed image based on the predicted deformation parameters.

In some embodiments, the predicted transformed image may be generated in a similar manner as how the preliminary transformed image is generated as described in operation 904. For example, the processing device 140 may generate the predicted transformed image by processing the sample image based on the predicted deformation parameters.

In 1008, the processing device 140 (e.g., the training module 830) may generate a parameter determination model by updating the initial model based on the predicted transformed image and the sample transformed image of each of the plurality of training samples.

The training of the initial model may include an iterative process. The plurality of training samples may be used to iteratively update model parameter(s) of the initial model until a termination condition is satisfied. Exemplary termination conditions may include that an image difference between the predicted transformed image and the sample transformed image is less than an image difference threshold value, a value of a loss function corresponding to the initial model is below a threshold value, a difference of values of the loss function obtained in a previous iteration and the current iteration is within a difference threshold value, a certain count of iterations has been performed, etc. For example, in a current iteration, an image difference between the predicted transformed image and a sample transformed image of each training sample may be determined, and the value of the loss function may be determined based on the image difference. If it is determined that the termination condition is satisfied in the current iteration (i.e., the value of the loss function is below a threshold value), the initial model may be designated as the parameter determination model; otherwise, the initial model may be further updated based on the value of the loss function.

By introducing the parameter determination model, the analysis of the big data may enable mining the complex relationship between feature information of the preliminary image and the deformation parameters, thus learning mechanism(s) of deformation parameter (e.g., the count and value(s)) setting. Therefore, the deformation parameters may be generated automatically, which can improve the efficiency and accuracy of the generation of the deformation parameters with respect to human or traditional determination approaches, and smooth the process of the image transformation, thereby improving the efficiency of the image transformation.

FIG. 11 is a flowchart illustrating an exemplary process 1100 for generating a preliminary transformed image according to some embodiments of the present disclosure. In some embodiments, the process 1100 may be performed to achieve at least part of operation 904 as described in connection with FIG. 9.

For the convenience of descriptions, an image coordinate system is introduced. Since the preliminary image, the preliminary transformed image, and the deformation map have the same size, they may correspond the same image coordinate system. A pixel in an image (e.g., the preliminary image, the preliminary transformed image, and a deformation map described below) may correspond to one coordinate in the image coordinate system. For illustration purposes, it is assumed that the images are 2D images and the image coordinate system is a 2D coordinate system.

In 1102, the processing device 140 (e.g., the generation module 820) may generate a plurality of deformation maps based on deformation parameters and a preliminary image.

A deformation map may be an image generated after an image transformation is performed based on a deformation parameter. In some embodiments, a size of each of the plurality of deformation maps may be the same as a size of the preliminary image. For example, if a height and a width of the preliminary image are H and W, respectively, a height and a width of each of the plurality of deformation maps may be H and W, respectively. The deformation map may include a deformed coordinate corresponding to a coordinate of each pixel in the preliminary image.

In some embodiments, for each of the deformation parameters, the processing device 140 may process the preliminary image based on the deformation parameter. For example, if the deformation parameters include m×n affine transformation matrices, the processing device 140 may perform the affine transformation on the preliminary image based on each of the m×n affine transformation matrices, so as to generate m×n deformation maps (i.e., affine transformation maps).

In some embodiments, the processing device 140 may normalize coordinates of the preliminary image to obtain normalized coordinates. A coordinate of the preliminary image refers to a coordinate of a pixel in the preliminary image in the image coordinate system. If a height and a width of the preliminary image are H and W, respectively, there are H×W coordinates or normalized coordinates of the preliminary image. Taking a two-dimensional preliminary image as an instance, each coordinate may include an abscissa in the image coordinate system (e.g., which corresponds to the width of the preliminary image) and an ordinate in the image coordinate system (e.g., which corresponds to the height of the preliminary image). The processing device 140 may normalize the abscissa of each coordinate into a target range (e.g., a range from −1 to 1) based on the width of the preliminary image. The processing device 140 may normalize the ordinate of each coordinate into the target range (e.g., a range from −1 to 1) based on the height of the preliminary image.

In some embodiments, the processing device 140 may process the normalized coordinates based on the deformation parameters to generate the plurality of deformation maps. For example, for each of the deformation parameters, the processing device 140 may multiply the normalized coordinates by the deformation parameter (e.g., an affine transformation matrix) to obtain deformed coordinates, and the deformed coordinates may form a deformation map (e.g., an affine transformation map) corresponding to the deformation parameter. In other words, the deformation map corresponding to a deformation parameter may include deformed coordinate of each coordinate of the preliminary image.

In 1104, for each of a plurality of coordinates in the image coordinate system, the processing device 140 (e.g., the generation module 820) may determine a deformation coordinate of the coordinate based on the plurality of deformation maps.

In some embodiments, the processing device 140 may determine whether a preset condition is satisfied based on the count of the deformation parameters and the size of the preliminary image. For example, the count of the deformation parameters may be m×n, and the size of the preliminary image may be H×W. When m is equal to H and n is equal to W, or m is equal to W and n is equal to H, the processing device 140 may determine that the preset condition is satisfied. In such cases, each coordinate of the preliminary image may have a corresponding deformation map. For each coordinate, the processing device 140 may determine a corresponding deformation map corresponding to the coordinate, and determine the deformation coordinate of the coordinate based on the deformation map corresponding to the coordinate.

Merely by way of example, if the height and the width of the preliminary image are 5, and a count of deformation parameters is 5×5 (i.e., 25), 25 deformation maps may be generated by processing the preliminary image based on each of the 25 deformation parameters, and each of the 25 deformation maps may include 25 coordinates. In such cases, each coordinate in the image coordinate system may have a corresponding deformation map, and a deformation coordinate of the coordinate may be determined based on the deformation map corresponding to the coordinate. For example, for an i-th coordinate in the image coordinate system, a corresponding deformation map may be an i-th deformation map, and a deformation coordinate of the i-th coordinate may be an i-th coordinate in the i-th deformation map. As used herein, i may be a positive integer, and i may be within a range from 1 to 25.

As another example, when m is not equal to H and/or n is not equal to W (e.g., the count of the deformation parameters is less than the count of pixels in the preliminary image), the processing device 140 may determine that the preset condition is not satisfied. The processing device 140 may divide the preliminary image into a plurality of image blocks. Each vertex of the plurality of image blocks may correspond to one of the plurality of deformation maps. In other words, the count of vertexes of the image blocks may be equal to the count of the deformation maps. For each coordinate in the image coordinate system, the processing device 140 may determine the deformation coordinate of the coordinate based on the plurality of image blocks and the plurality of deformation maps.

For example, the processing device 140 may determine first coordinates and second coordinates among the plurality of coordinates in the image coordinate system. Each first coordinate may correspond to a vertex of the plurality of image blocks, and the second coordinates may be coordinates other than the first coordinates. The processing device 140 may determine the deformation coordinates of the first coordinates based on the plurality of deformation maps, and determine the deformation coordinate of each second coordinate based on at least part of the deformation coordinates of the first coordinates. For example, the processing device 140 may determine the deformation coordinate of each second coordinate by using an interpolation algorithm based on at least part of the deformation coordinates of the first coordinates adjacent to the second coordinate.

Merely by way of example, referring to FIG. 12 again, a height and a width of a preliminary image are 5. When a count of deformation parameters is 9 (i.e., 3×3), the preliminary image may be divided into 4 image blocks (as indicated by solid blocks), and 9 deformation maps may be generated based on the 9 deformation parameters and the preliminary image. Corresponding, the preliminary transformed image may include 9 first coordinates (i.e., coordinates corresponding to 9 vertices of the 4 image blocks), and the preliminary transformed image may include 16 second coordinates. Each of the 9 first coordinates may correspond to one of the 9 deformation maps. Deformation coordinates of the 9 first coordinates may be determined based on the 9 deformation maps, and deformation coordinates of the 16 second coordinates may be determined based on the deformation coordinates of the 9 first coordinates.

In some embodiments, the processing device 140 may determine whether a coordinate in the image coordinate system is a first coordinate by determining whether a coordinate corresponds to a vertex of the plurality of image blocks. For example, if a coordinate of a coordinate is (x, y), a count of deformation parameters is n×n, and a count of image blocks is p×p, the processing device 140 may determine whether a remainder of x and p is equal to 0 and a remainder of y and p is equal to 0. If the remainder of x and p is equal to 0 and the remainder of y and p is equal to 0, the processing device 140 may determine that the coordinate in the image coordinate system is a first coordinate or the coordinate and corresponds to a vertex of the plurality of image blocks. If the remainder of x and p is not equal to 0 or the remainder of y and p is not equal to 0, the processing device 140 may determine the coordinate in the image coordinate system is a second coordinate.

In some embodiments, the processing device 140 may determine a deformation map corresponding to a first coordinate according to a positioning function:

$$k = int ( n \times ( \frac{y}{p} ) + ( \frac{x}{p} ) ) ,$$

where int represents a rounding operation, and k represents a serial number of the deformation maps. That is, the k-th deformation map may correspond to the first coordinate. Then, the deformation coordinate of the first coordinate may be determined based on the deformation map corresponding to the first coordinate.

After the deformation coordinates of the first coordinates are determined, the deformation coordinate of each second coordinate may be determined based on at least part of the deformation coordinates of the first coordinates. For example, referring to FIG. 12 again, points A and C are first coordinates, and a point B is a second coordinate. After deformation coordinates of the points A and C are determined, a deformation coordinate of the point B may be determined based on the deformation coordinates of the points A and C by, for example, performing an interpolation operation on the deformation coordinates of the points A and C.

In 1106, for each of the plurality of deformation coordinates corresponding to the coordinates, the processing device 140 (e.g., the generation module 820) may determine a pixel value of the deformation coordinate based on the preliminary image.

In some embodiments, the processing device 140 may scale a deformation coordinate based on a size of the preliminary image and a size of the preliminary transformed image. For example, if a height and a width of the preliminary image are H and W, respectively, the processing device 140 may scale the deformation coordinate into a range of [0, H] and [0, W] to obtain a scaled coordinate. If the scaled coordinate is an integer coordinate, the processing device 140 may designate a pixel value corresponding the scaled coordinate in the preliminary image as the pixel value of the deformation coordinate. If the scaled coordinate is not an integer coordinate, the processing device 140 may determine the pixel value of the deformation coordinate based on pixel values of coordinates adjacent to the scaled coordinate in the preliminary image. For instance, the processing device 140 may determine the pixel value of the deformation coordinate by performing an interpolation operation on the pixel values of the coordinates adjacent to the scaled coordinate. Merely by way of example, a grid sample function in a pytorch framework may be used to determine the pixel value of the deformation coordinate.

In 1108, the processing device 140 (e.g., the generation module 820) may generate a preliminary transformed image based on the plurality of deformation coordinates and their respective pixel values.

For example, for each deformation coordinate, the preliminary transformed image may include a pixel that is located at the deformation coordinate and have the pixel value corresponding to the deformation coordinate.

In some embodiments, the processing device 140 may obtain an initialized image. A size of the initialized image may be the same as the size of the preliminary image, and a pixel value of each coordinate in the initialized image may be 0. And then, each pixel in the initialized image may represent a coordinate in the image coordinate system, the processing device 140 may determine the deformation coordinate of the pixel by performing operation 1104, and determine the pixel value of the pixel by performing operation 1106. Therefore, the initialized image may be transformed into the preliminary transformed image based on the deformation coordinate and the pixel value of each pixel in the initialized image.

According to some embodiments of the present disclosure, by generating the plurality of deformation maps, the deformation coordinate and the pixel value of each of the plurality of coordinates in the image coordinate system may be determined. Correspondingly, the preliminary transformed image may be generated. Therefore, different deformations can be performed on different portions of the preliminary image, and image distortion and edge blurring after deformation can be reduced.

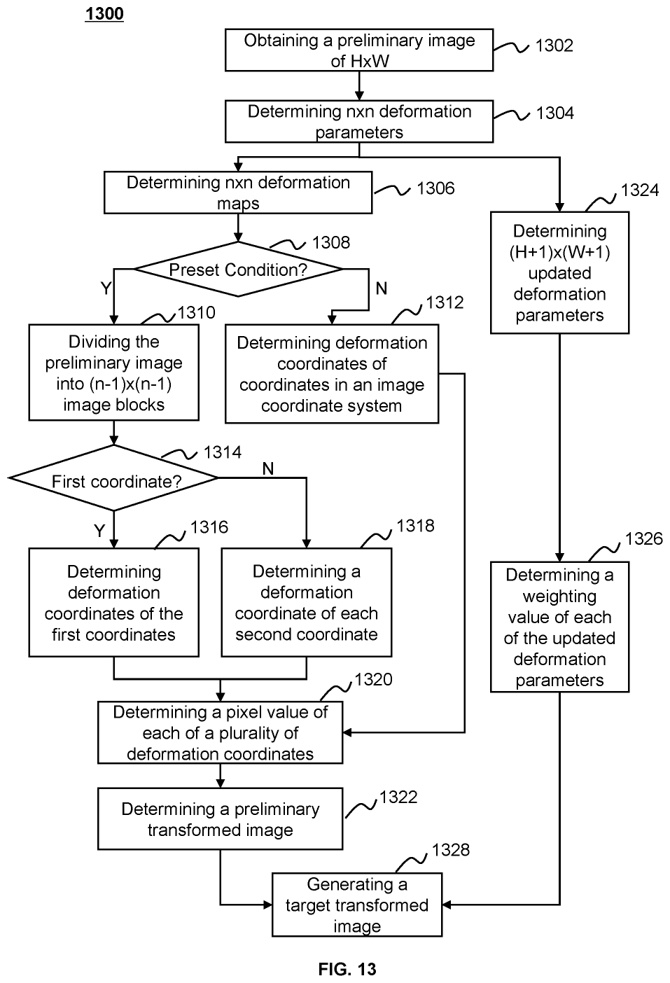

FIG. 13 is a schematic diagram illustrating an exemplary process 1300 for image transformation according to some embodiments of the present disclosure.

In 1302, the processing device 140 may obtain a preliminary image. A height and a width of the preliminary image may be H and W, respectively.

In 1304, the processing device 140 may determine deformation parameters of the preliminary image. A count of the deformation parameters may be n×n. The deformation parameters may be the same or different.

In 1306, the processing device 140 may determine deformation maps based on the deformation parameters and the preliminary image. A count of the deformation parameters may be n×n.

In 1308, the processing device 140 may determine whether a preset condition is satisfied. When n is not equal to H or W, the processing device 140 may determine that the preset condition is satisfied. When n is equal to H and W, the processing device 140 may determine that the preset condition is not satisfied.

If the preset condition is satisfied, the process 1300 may proceed to operation 1310. If the preset condition is not satisfied, the process 1300 may proceed to operation 1312.

In 1310, the processing device 140 may divide the preliminary image into a plurality of image blocks. A count of the plurality of image blocks may be (n−1)×(n−1).

In 1312, the processing device 140 may determine deformation coordinates of coordinates in an image coordinate system. For each coordinate, the processing device 140 may determine a corresponding deformation map of the coordinate, and determine the deformation coordinate of the coordinate based on the deformation map corresponding to the coordinate.

In 1314, for each coordinate in the image coordinate system, the processing device 140 may determine whether the coordinate in the image coordinate system is a first coordinate.

If the coordinate in the image coordinate system is the first coordinate, the process 1300 may proceed to operation 1316. If the coordinate in the image coordinate system is not the first coordinate, the process 1300 may proceed to operation 1318.

In 1316, the processing device 140 may determine deformation coordinates of the first coordinates based on the plurality of deformation maps.

In 1318, the processing device 140 may determine a deformation coordinate of each second coordinate based on at least part of the deformation coordinates of the first coordinates.

In 1320, the processing device 140 may determine a pixel value of each of a plurality of deformation coordinates of the plurality of coordinates based on the preliminary image.

In 1322, the processing device 140 may generate a preliminary transformed image based on the plurality of deformation coordinates and their respective pixel values.

In 1324, the processing device 140 may determine updated deformation parameters based on the deformation parameters. A count of the updated deformation parameters may be H×W.

In 1326, the processing device 140 may determine a weighting value of each of the updated deformation parameters. The weighting value may relate to a proportion of a deformed region in the preliminary image when the preliminary image is transformed based on the updated deformation parameter.

In 1328, the processing device 140 may generate a target transformed image by processing the preliminary transformed image based on the weighting value of each of the updated deformation parameters.

It should be noted that the descriptions of the processes 300, 600, 700, 900-1100, and 1300 are provided for the purposes of illustration, and are not intended to limit the scope of the present disclosure. For persons having ordinary skills in the art, various variations and modifications may be conducted under the teaching of the present disclosure. For example, the processes 300, 600, 700, 900-1100, and 1300 may be accomplished with one or more additional operations not described, and/or without one or more of the operations discussed. Additionally, the order in which the operations of the processes 300, 600, 700, 900-1100, and 1300 is not intended to be limiting. However, those variations and modifications may not depart from the protection of the present disclosure.

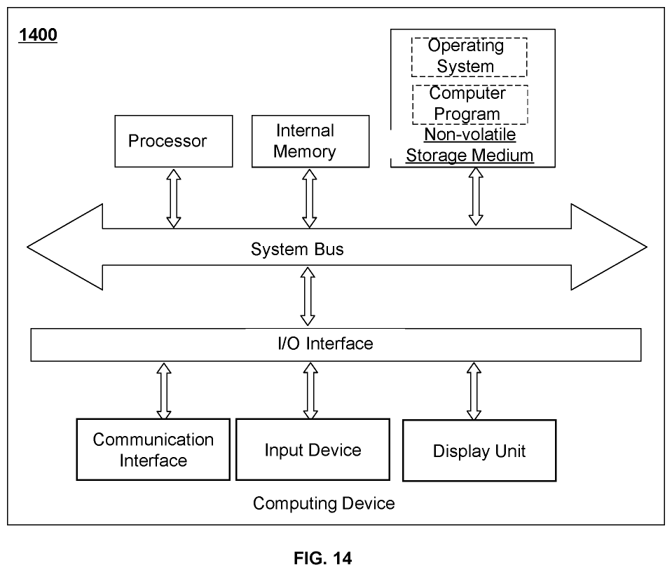

FIG. 14 is a schematic diagram illustrating an exemplary computing device 1400 according to some embodiments of the present disclosure.

In some embodiments, one or more components of the imaging system 100 may be implemented on the computing device 1400. For example, a processing engine may be implemented on the computing device 1400 and configured to implement the functions and/or methods disclosed in the present disclosure.

The computing device 1400 may include any components used to implement the imaging system 100 described in the present disclosure. For example, the processing device 140 may be implemented through hardware, software program, firmware, or any combination thereof, on the computing device 1400. For illustration purposes, only one computer is described in FIG. 14, but computing functions related to the imaging system 100 described in the present disclosure may be implemented in a distributed fashion by a group of similar platforms to spread the processing load of the imaging system 100.

The computing device 1400 may include a communication port connected to a network to achieve data communication. The computing device 1400 may include a processor (e.g., a central processing unit (CPU)), a memory, a communication interface, a display unit, and an input device connected by a system bus. The processor of the computing device 1400 may be used to provide computing and control capabilities. The memory of the computing device 1400 may include a non-volatile storage medium, an internal memory. The non-volatile storage medium may store an operating system and a computer program. The internal memory may provide an environment for the execution of the operating system and the computer program in the non-volatile storage medium. The communication interface of the computing device 1400 may be used for wired or wireless communication with an external terminal. The wireless communication may be realized through Wi-Fi, a mobile cellular network, a near field communication (NFC), etc. When the computer program is executed by the processor, a method for determining feature points may be implemented. The display unit of the computing device 1400 may include a liquid crystal display screen or an electronic ink display screen. The input device of the computing device 1400 may include a touch layer covered on the display unit, a device (e.g., a button, a trackball, a touchpad, etc.) set on the housing of the computing device 1400, an external keyboard, an external trackpad, an external mouse, etc.

Merely for illustration, only one processor is described in FIG. 14. However, it should be noted that the computing device 1400 in the present disclosure may also include multiple processors. Thus operations and/or method steps that are performed by one processor as described in the present disclosure may also be jointly or separately performed by the multiple processors. For example, if the processor of the computing device 1400 in the present disclosure executes both operation A and operation B, it should be understood that operation A and operation B may also be performed by two or more different processors jointly or separately (e.g., a first processor executes operation A and a second processor executes operation B, or the first and second processors jointly execute operations A and B).

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for image processing, implemented on a computing device having at least one processor and at least one storage device, the method comprising:
obtaining original projection data of a target subject;
for each of at least one slice location of the target subject, generating a plurality of candidate slice images of the slice location based on a plurality of distance-weight relationships and the original projection data, each of the plurality of distance-weight relationships indicating a weight of a portion of the original projection data acquired by a radiation source and a distance from the radiation source to the slice location when acquiring the portion of the original projection data; and
generating at least one target medical image of the target subject based on the plurality of candidate slice images of each of the at least one slice location.

2. The method of claim 1, wherein the plurality of distance-weight relationships of a slice location are generated by:
determining an initial distance-weight relationship corresponding to the slice location; and
determining the plurality of distance-weight relationships by translating the initial distance-weight relationship.

3. The method of claim 2, wherein the initial distance-weight relationship corresponding to the slice location is determined based on at least one of feature information of the slice location or a moving speed of the radiation source with respect to the target subject.

4. The method of claim 2, wherein the translating the initial distance-weight relationship includes:
determining motion information of the target subject during the acquisition of the original projection data; and
translating the initial distance-weight relationship based on the motion information.

5. The method of claim 1, wherein the generating at least one target medical image of the target subject based on the plurality of candidate slice images of each of the at least one slice location includes:
for each of the at least one slice location, determining a target slice image from the plurality of candidate slice images of the slice location; and
generating a target 3D image of the target subject based on the target slice image of each of the at least one slice location.

6. The method of claim 1, wherein the generating at least one target medical image of the target subject based on the plurality of candidate slice images of each of the at least one slice location includes:
generating a plurality of candidate 3D images of the target subject based on the plurality of candidate slice images of each of the at least one slice location;
obtaining an evaluation score of each of the plurality of candidate 3D images by evaluating each of the plurality of candidate 3D images; and
determining a target 3D image from the plurality of candidate 3D images based on the plurality of evaluation scores.

7. The method of claim 6, wherein the generating at least one target medical image of the target subject based on the plurality of candidate slice images of each of the at least one slice location includes:
determining deformation parameters of the target 3D image;
generating a preliminary transformed image by processing the target 3D image based on the deformation parameters;
determining updated deformation parameters based on the deformation parameters, the count of the updated deformation parameters being determined based on the size of the target 3D image; and
generating the at least one target medical image by processing the preliminary transformed image based on the updated deformation parameters.

8. The method of claim 7, wherein the deformation parameters of the target 3D image are determined using a parameter determination model, and the parameter determination model is a trained machine learning model.

9. A system for image processing, comprising:
at least one storage device including a set of instructions; and
at least one processor configured to communicate with the at least one storage device, wherein when executing the set of instructions, the at least one processor is configured to direct the system to perform operations including:
obtaining original projection data of a target subject;
for each of at least one slice location of the target subject, generating a plurality of candidate slice images of the slice location based on a plurality of distance-weight relationships and the original projection data, each of the plurality of distance-weight relationships indicating a weight of a portion of the original projection data acquired by the radiation source and a distance from the radiation source to the slice location when acquiring the portion of the original projection data; and
generating at least one target medical image of the target subject based on the plurality of candidate slice images of each of the at least one slice location.

10. A method for image processing, implemented on a computing device having at least one processor and at least one storage device, the method comprising:
determining deformation parameters of a preliminary image;
generating a preliminary transformed image by processing the preliminary image based on the deformation parameters;
determining updated deformation parameters based on the deformation parameters; and
generating a target transformed image by processing the preliminary transformed image based on the updated deformation parameters.

11. The method of claim 10, wherein the deformation parameters of the preliminary image are determined using a parameter determination model, and the parameter determination model is a trained machine learning model.

12. The method of claim 11, wherein the parameter determination model is generated by:
obtaining a plurality of training samples, each of the plurality of training samples including a sample image and a sample transformed image corresponding to the sample image;
for each of the plurality of training samples,
generating predicted deformation parameters by inputting the sample image of the training sample into an initial model; and
generating a predicted transformed image by transforming the sample image of the training sample based on the predicted deformation parameters; and
generating the parameter determination model by updating the initial model based on the predicted transformed image and the sample transformed image of each of the plurality of plurality of training samples.

13. The method of claim 10, wherein the generating a preliminary transformed image by processing the preliminary image based on the deformation parameters includes:
generating a plurality of deformation maps based on the deformation parameters and the preliminary image;
for each of a plurality of coordinates in an image coordinate system, determining a deformation coordinate of the coordinate based on the plurality of deformation maps;
determining a pixel value of each of a plurality of deformation coordinates of the plurality of coordinates based on the preliminary image; and
generating the preliminary transformed image based on the plurality of deformation coordinates and their respective pixel values.

14. The method of claim 13, wherein for each coordinate, the determining a deformation coordinate of the coordinate based on the plurality of deformation maps includes:
determining whether a preset condition is satisfied based on the count of the deformation parameters and the size of the preliminary image; and
in response to determining that the preset condition is satisfied,
for each coordinate, determining a corresponding deformation map corresponding to the coordinate, and determining the deformation coordinate of the coordinate based on the deformation map corresponding to the coordinate.

15. The method of claim 13, wherein for each coordinate, the determining a deformation coordinate of the coordinate based on the plurality of deformation maps includes:
determining whether a preset condition is satisfied based on the count of the deformation parameters and the size of the preliminary image;
in response to determining that the preset condition is not satisfied, dividing the preliminary image into a plurality of image blocks, each vertex of the plurality of image blocks corresponding to one of the plurality of deformation maps; and
for each coordinate, determining the deformation coordinate of the coordinate based on the plurality of image blocks and the plurality of deformation maps.

16. The method of claim 15, wherein for each coordinate, the determining the deformation coordinate of the coordinate based on the plurality of image blocks and the plurality of deformation maps includes:
determining first coordinates and second coordinates among the plurality of coordinates, each first coordinate being corresponding to a vertex of the plurality of image blocks, the second coordinates being coordinates other than the first coordinates;
determining the deformation coordinates of the first coordinates based on the plurality of deformation maps; and
determining the deformation coordinate of each second coordinate based on at least part of the deformation coordinates of the first coordinates.

17. The method of claim 10, wherein the count of the updated deformation parameters being determined based on the size of the preliminary image.

18. The method of claim 17, wherein the determining updated deformation parameters based on the deformation parameters includes:
determining the updated deformation parameters by performing interpolation operation on the deformation parameters based on the size of the preliminary image.

19. The method of claim 18, wherein the generating a target transformed image by processing the preliminary transformed image based on the updated deformation parameters includes:
determining a weighting value of each of the updated deformation parameters, the weighting value relating to a proportion of a deformed region in the preliminary image when the preliminary image is transformed based on the updated deformation parameter; and
generating the target transformed image by processing the preliminary transformed image based on the weighting value of each of the updated deformation parameters.

20. The method of claim 10, wherein the preliminary image is obtained by obtaining original projection data of a target subject;
for each of at least one slice location of the target subject, generating a plurality of candidate slice images of the slice location based on a plurality of distance-weight relationships and the original projection data, each of the plurality of distance-weight relationships indicating a weight of a portion of the original projection data acquired by the radiation source and a distance from the radiation source to the slice location when acquiring the portion of the original projection data; and
generating the preliminary image of the target subject based on the plurality of candidate slice images of each of the at least one slice location.
